(19) United States
(12) Patent Application Publication
(10) Pub. No.: US 2026/0098054 A1
(43) Pub. Date: Apr. 9, 2026

(54) COMPOSITIONS AND METHODS FOR CONSTRUCTING CONJUGATES

(71) Applicant: THE UNIVERSITY OF HONG KONG, Hong Kong (CN)

(72) Inventors: Xuechen LI, Hong Kong (CN); Hey Pui CHEUNG, Hong Kong (CN)

(21) Appl. No.: 19/103,950

(22) PCT Filed: Aug. 22, 2023
(86) PCT No.: PCT/CN2023/114213

§ 371 Date: Feb. 14, 2025

Related U.S. Application Data
(60) Provisional application No. 63/401,301, filed on Aug. 26, 2022.

(51) Int. Cl. C07K 1/107; C07K 1/04

(52) U.S. Cl. CPC C07K 1/1075; C07K 1/04; C07K 1/1077

(57) ABSTRACT

Disclosed are methods for constructing conjugates. An amine-containing component and a thiol-containing component, in comparable mole ratios, react with an ortho-phthalaldehyde in one step, to generate a conjugate bearing a substituted or an unsubstituted 1-thio-isoindole structure in the presence of an additive. The method provides a platform for the efficient and practical constructions of bioactive modular motifs.

### CROSS-REFERENCE TO RELATED APPLICATIONS

This application claims benefit of and priority to U.S. Ser. No. 63/401,301, filed Aug. 26, 2022, which is incorporated herein by reference in its entirety.

### FIELD OF THE INVENTION

The disclosed invention is generally in the field of conjugates and methods of constructing conjugates, and particularly in the area of conjugates containing a 1-thiol-isoindole structure that are formed by reacting an amine-containing component and a thiol-containing component with an ortho-phthalaldehyde, preferably in one-step.

### BACKGROUND OF THE INVENTION

Structures constructed with different modular motifs are attracting increasing interests in drug discovery, especially in the field of targeted-cancer therapy. Conjugates endowed with a cytotoxic agent and disease-targeting function have been designed to localize the pharmacological activity to tumor cells and maximize the cytotoxic agent's therapeutic index (Srinivasarao &Low, Chem. Rev. 2017, 117, 12133-12164). For example, antibody-drug conjugates and peptide-drug conjugates have rapidly emerged as promising therapeutic modalities, due to their enhanced local efficacy and reduced peripheral toxicity (Thomas, et al, Lancet Oncol. 2016, 17, 254-262; Cooper, et al., Chem. Soc. Rev. 2021, 50, 1480-1494). Despite the attractiveness of these structural conjugate motifs, the chemistry to link different modular motifs is not straightforward. First, the conjugation reaction between the different modalities (such as the drug and the targeting agent) must be chemoselective and effective. Further, all other functional groups present in these moieties should not react, otherwise orthogonal protecting groups are needed.

Different chemical linkers such as amide, hydrazone, triazoles, and disulfide bonds have been reported for peptide/protein bioconjugation (Alas, et al., J. Med. Chem. 2020, 64, 216-232). Nevertheless, new linker strategies are continuously being explored, to expand the current toolbox for conjugation, including bioconjugation. The development of chemoselective reactions and modular strategies will be of great value for constructing conjugates efficiently.

Ortho-phthalaldehyde (OPA) has gained renascences for protein bioconjugation and peptide cyclization. Roth first made a seminal finding that OPA could serve as a reagent for fluorometric detection of α-amino acids in the presence of 2-mercaptoethanol (Roth, Anal. Chem. 1971, 43, 880-882). The strongly fluorescent product generated from this OPA-amine-thiol reaction was later characterized as 1-thio-isoindole. However, unlike the single amino acids used in Roth, peptides showed greatly reduced fluorescence for unclear reasons at the time. Subsequently, Roth's method was modified by Benson in 1975 (Benson &Hare, Proc. Natl. Acad. Sci. U.S.A. 1975, 72, 619-622). The new protocol made use of excess 2-mercaptocthanol and Brij® to react with OPA in boric buffer (pH 9.7), prior to the addition of amino acids. Since then, this optimized method has been widely adopted for amino acid detection, attributed to its improved effectiveness and reproducibility. In addition, conditions that utilize the thiol groups in several orders of magnitude excess have also been frequently adopted (Zuman, Chem. Rev. 2004, 104, 3217-3238). Meanwhile, the successful detection of peptides and proteins was also reported, by adding 2-mercaptoethanol in 189-fold excess compared to OPA (Weidekamm, et al., Anal. Biochem. 1973, 54, 102-114). Nevertheless, the underlying reason for the large excess amounts of thiol needed in these reactions remained unclear.

In this endeavor, an OPA reaction with the amine groups on peptides/proteins to form phthalimidines (isoindolin-1-ones), in aqueous buffer conditions have been explored (Tung, et al., Org. Lett. 2016, 18, 2569-2578). As opposed to the conditions reported by previous studies which involved organic solvents (Thiele &Schneider, Liebigs Annalen 1909, 369, 287-299; Takahashi &Hatanaka, Heterocycles 1997, 45, 2475-2499; Pérard-Viret, et al., Tetrahedron 2002, 58, 5103-5108; Kulla &Zuman, Org. Biomol. Chem. 2008, 6, 3771-3780), the reaction rate dramatically increased in aqueous buffer (pH 6-11) (Tung, et al., Org. Lett. 2016, 18, 2569-2578). Moreover, this aqueous OPA-amine two-component reaction led to rapid and quantitative conversions, making it promising for protein/peptide bioconjugation. Mechanistically, the discovery helped explain the previous findings about the requirement for large excess amounts of the thiol group: the OPA-amine two-component reaction competes with the OPA-amine-thiol three-component reaction in aqueous buffers, requiring excess amounts of the thiol group to ensure the formation of the desired three-component product. This finding is consistent with a recent study (Nie, et al., J. Org. Chem. 2022, 87, 2551-2558). The reaction between OPA, an amine-containing DNA, and 4-tert-butylbenzenethiol was reported to generate a mixture of two-component and three-component products. Notably, 300-3000 equivalents of the thiol compound were added, and the conversion towards the three-component product was 85%. Further increasing the thiol compound to 3000 equivalents improved the conversion up to 95%.

To compromise the highly efficient OPA-amine two-component intermolecular reactions, research groups have independently reported the intramolecular OPA-amine-thiol reaction for peptide cyclization (Zhang, et al., J. Am. Chem. Soc. 2019, 141, 12274-12279; Todorovic, et al., Angew. Chem., Int. Ed. 2019, 58, 14120-14124). In this intramolecular setting, an unprotected peptide bearing both the amine and thiol groups rapidly reacts with OPA to undergo thiol-amine cyclization, much faster than the OPA-amine two-component reaction. Hence, only the desired three-component product was generated. In addition, an intramolecular OPA-amine-amine reaction for peptide stapling has been recently reported (Li, et al., Nat. Commun. 2022, 13, 311). However, a major concern with intramolecular reactions is that in nature and most chemical and/or biochemical syntheses settings, the reactants (containing the thiol or amine groups) are not usually present or are not purchased in their intramolecular forms, i.e., where both reacting groups present in the same molecule.

Although the intramolecular OPA reaction with peptides is simple, rapid, and chemoselective, it remains to be determined whether this reaction can be re-shifted intermolecularly, and particularly for bioactive modular constructions. One major existing hurdle is that traditionally a large excess of the thiol group is needed to achieve the three-component product. For bioconjugation, the peptides, proteins, and drugs that are used are often obtained in limited quantities and at a high economic cost. Therefore, it is cost-prohibitive and unaffordable to react these components in large excess amounts. Another problem is that the conjugate's core (such as the 1-thio-isoindole product obtained from the OPA-amine-thiol reaction) suffers from low stability. Hence, there remains a need to develop improved, practical, and efficient approaches for intermolecular reactions of modular motifs to produce conjugations, such as bioconjugates.

Therefore, it is an object of the invention to provide improved methods for the intermolecular production of conjugates.

It is also an object of the invention to provide improved methods for the intermolecular production of bioconjugates, such as those that contain a biomolecule, including peptides, carbohydrates, proteins, lipids, and nucleic acids.

### BRIEF SUMMARY OF THE INVENTION

Disclosed are conjugates, such as bioconjugates, and methods of making the conjugates via intermolecular reactions between modular motifs in an aqueous buffer. The method involves reacting a first reactant having a structure of Formula I:

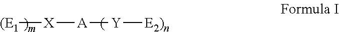

with (i) a second reactant containing a thiol nucleophile and (ii) a third reactant containing an amine nucleophile, in the presence of an additive to form a conjugate, preferably wherein the additive suppresses a product formed by a reaction between only the first reactant and the third reactant,
wherein:
A is a substituted aryl, an unsubstituted aryl, a substituted heteroaryl, an unsubstituted heteroaryl, a substituted polyaryl, an unsubstituted polyaryl, a substituted polyheteroaryl, an unsubstituted polyheteroaryl, preferably a substituted aryl or an unsubstituted aryl,
preferably, m and n are 1,
preferably, X and Y are absent,
preferably, E₁ and E₂ are vicinal in the first reactant, such that they are directly attached to adjacent atoms in a chemical ring of the first reactant, and
preferably E₁ and E₂ are aldehydes.

Preferably, the first reactant is an ortho-phthalaldehyde (OPA).

Traditionally, these intermolecular reactions have involved using a large excess (such as at least 4.6-fold equivalents) of the reactant containing the thiol group, to ensure the formation of the desired product containing both reactants in a chemoselective manner. However, the use of the reactant in large excess renders the reaction inefficient and cost-prohibitive for bioconjugation. As described below, the presence of an additive reduces or eliminates the need to utilize a large excess of the reactant containing a thiol nucleophile. Preferably, the additive, such as guanidine in its salt form (such as a guanidium, e.g., guanidium chloride) generates an OPA-guanidine adduct that suppresses the formation of imine-aldehyde that is a common intermediate in the non-selective formation of two-component and three-component products. Thus, preferably, the mole ratio of the reactant containing the thiol group and the reactant containing the amine group is between 1:4 to 4:1.

In addition, the 1-thio-isoindole product obtained from the OPA-amine-thiol reaction suffers from low stability. Preferably, the low stability problem is alleviated by using a sterically hindered thiol group that reacts with the OPA and/or the incorporation of substituents on the heteroaryl group of the 1-thio-isoindole core.

Preferably, forming the conjugate involves:
(i) adding an additive in a suitable buffer, such as PBS, to form a solution, dispersion, or suspension,
(ii) adding a first reactant, such as OPA, and a second reactant, such as a reactant containing a thiol group in the solution, dispersion, or suspension of step (i) to form another solution, dispersion, or suspension, and
(iii) adding a third reactant containing an amine group to the solution of step (ii) to form another solution, dispersion, or suspension.

Preferably, the conjugate has a structure:

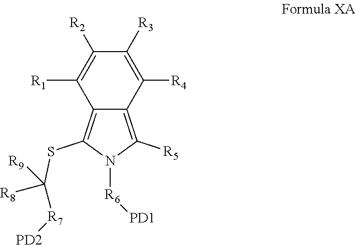

wherein:
R₁-R₄ are hydrogen or at least one of R₁-R₄ independently contains a targeting agent, diagnostic agent, prophylactic agent, or a therapeutic agent, preferably wherein the targeting agent contains a peptide,
R₅ is hydrogen, unsubstituted C₁-C₁₀ alkyl, substituted C₁-C₁₀ alkyl, unsubstituted C₁-C₁₀ alkenyl, or substituted C₁-C₁₀ alkenyl,
R₆ and R⁷ are independently unsubstituted C₁-C₁₀ alkyl, or substituted C₁-C₁₀ alkyl, with the proviso that R₆ and R⁷ are at least divalent, as valency permits, optionally wherein R⁶ and R⁷ independently contain a stimuli-responsive chemical moiety, wherein preferably R₆ contains the optional stimuli-responsive chemical moiety,
R₈ and R₉ are independently hydrogen, unsubstituted C₁-C₁₀ alkyl, substituted C₁-C₁₀ alkyl, with the proviso that at least one of R₈ and R₉ is not hydrogen, such as unsubstituted C₁-C₁₀ alkyl, or substituted C₁-C₁₀ alkyl, preferably wherein R₈ and R₉ are independently unsubstituted C₁-C₁₀ alkyl or substituted C₁-C₁₀ alkyl, and
(i) PD1 and PD2 contain a peptide, preferably an unprotected peptide, or
(ii) one of PD1 and PD2 contains a peptide, preferably an unprotected peptide, and the other of PD1 and PD2 contains a non-peptide therapeutic agent, non-peptide diagnostic agent, non-peptide prophylactic agent, or non-peptide targeting agent, preferably wherein one of PD1 and PD2 contains a peptide, preferably an unprotected peptide, and the other of PD1 and PD2 contains a non-peptide therapeutic agent.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIG. 1 is a schematic showing the synthesis of a conjugate from a first reactant (such as ortho-phthalaldehyde (OPA)), a second reactant (such as a molecule containing a thiol group), and a third reactant (such as a molecule containing an amine group).

FIGS. 2A-2D are schematics of OPA-based reactions. FIG. 2A shows the OPA-amine-thiol intermolecular three-component reaction reported by Roth (Roth, Anal. Chem. 1971, 43, 880-882), Benson (Benson &Hare, Proc. Natl. Acad. Sci. U.S.A. 1975, 72, 619-622), and Weidekamm (Weidekamm, Anal. Biochem. 1973, 54, 102-114). FIG. 2B shows a schematic of the OPA-amine two-component reaction reported by Thiele (Thiele &Schneider, Liebigs Annalen 1909, 369, 287-299) and Li (Tung, et al., Org. Lett. 2016, 18, 2569-2578). FIG. 2C shows a schematic of the OPA-amine-thiol intramolecular three-component reaction reported by Li (Zhang, ct al., J. Am. Chem. Soc. 2019, 141, 12274-12279) and Perin (Todorovic, et al., Angew. Chem., Int. Ed. 2019, 58, 14120-14124). FIG. 2D shows a guanidine-additive facilitated intermolecular three-component reaction using stoichiometric amount of the thiol component described in this work. “1°-thiol” and “3°-thiol” refer to a primary thiol and a tertiary thiol, respectively.

FIGS. 3A-3Q show intermolecular OPA-amine-thiol three-component reactions for the construction of peptide-peptide conjugates, and some peptide-peptide conjugates. FIG. 3A shows peptide sequences of a thiol-containing peptide and an amine-containing peptide. FIG. 3B shows a control reaction, Condition [A], without an additive, such as guanidine. FIG. 3C shows a liquid chromatography trace of the crude reaction mixture of PPC-1 without an additive. FIG. 3D shows a test reaction, Condition [B], in the presence of an additive, such as guanidine. FIG. 3E shows a liquid chromatography trace of the crude reaction mixture of PPC-1 with an additive, such as guanidine. FIGS. 3F-3Q show representative sequences of PPC 1-12 and their respective isolated yields. The concentration of the additive, such as guanidine, applied ranged from 3 M to 8 M. The isolated yield was calculated based on the HPLC purified product.

FIG. 4A is a schematic of a three-component conjugate containing a 1-thio-isoindole core. FIG. 4B is a schematic of a further-functionalized conjugate containing a 1-thio-isoindole core. As shown, the linker between the drug and the 1-thio-isoindole core contains a stimuli-responsive bond (such as a disulfide bond). FIGS. 4C-4J are examples of bioconjugates produced using the methods described herein. Reaction conditions are as follows: [C]: thiol (1.0 equiv.), amine (1.5 equiv.), OPA (1.2 equiv.) in PBS buffer pH 7.4 with 1 M guanidine, 0.5 mM, room temperature, 1 h; [D]: [C], one-point DMAC (2.0 equiv.), 1 h; [E]: 3°-thiol (1.0 equiv.), OPA (1.2 equiv.) in PBS buffer pH 7.4 with 6 M guanidine, 5 mM, room temperature, 3 h; [F]: 3°-thiol (3.0 equiv.), amine (1.0 equiv.), functionalized OPA-fluorescein (3.0 equiv.) in PBS buffer pH 7.4 with 6 M guanidine, 1 mM 37° C., 2 h; [G] 3°-thiol (2.0 equiv.), amine (1.0 equiv.), functionalized OPA-peptide (1.2 equiv.) in PBS buffer pH 7.4 with 6 M guanidine, 1 mM room temperature, 2 h. FIG. 4K shows the conjugation of a cytotoxic agent and a targeting agent using OPA to form a conjugate PDC-1, and a liquid chromatography trace of the crude reaction mixture of PDC-1. “3°-thiol” refers to a tertiary thiol.

FIGS. 5A-5C are line graphs of stability test and biological evaluations of PDC 1-6. FIG. 5A shows stability tests of PDC-1 and PDC-3 dissolved in 5% ACN in H2 O or 5% ACN in PBS buffer pH 7.4, at a concentration of 0.5 mM and placed at room temperature or 37° C. as indicated. The compound was checked by LCMS after 24 h and 48 h. FIG. 5B shows the results of a CCK-8 assay in U87-MG cells treated with PDCs 1-6. FIG. 5C shows the results of a CCK-8 assay in HEK-293T cells treated with PDCs 1-6.

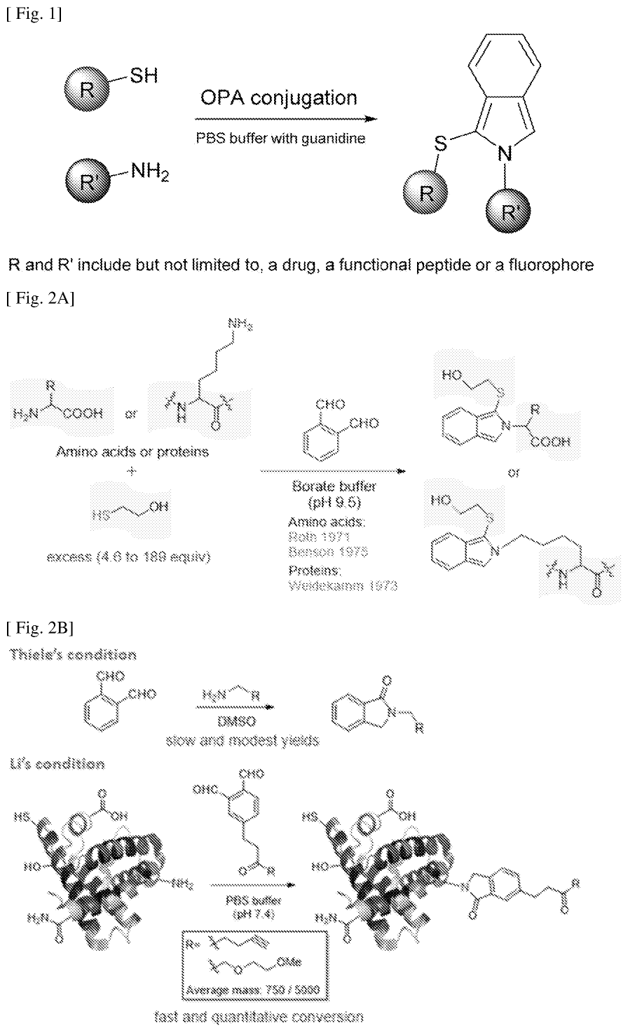

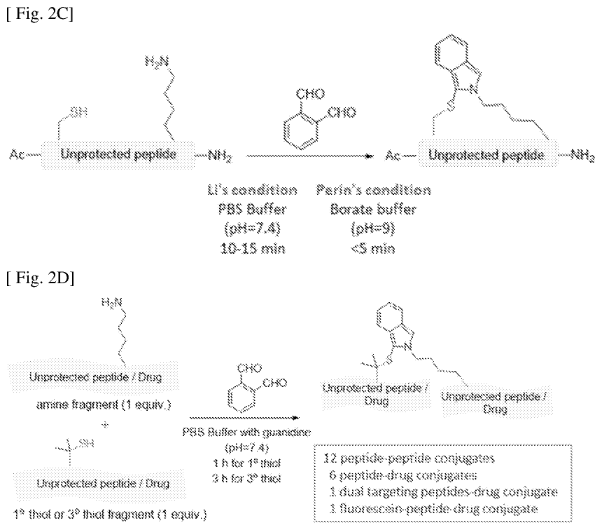

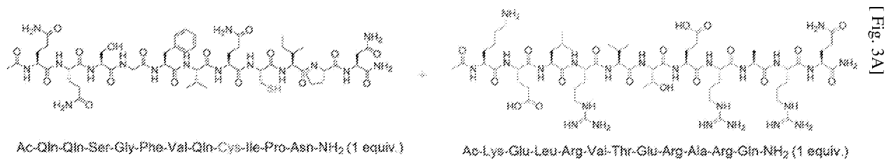

### DETAILED DESCRIPTION OF THE INVENTION

### I. Definitions

“Additive,” as used herein, refers to a compound that suppresses an undesirable side reaction pathway in the reaction, while not hindering progress of another desired reaction pathway. For instance, the additive associates with a reactant in a chemical reaction and suppresses the reaction between that reactant and another reactant, as compared to a different reactant, involved in the chemical reaction. For example, the additive can suppress a reaction pathway that leads to the formation of a product that involves the reaction between only an amine group and an electrophile in another reactant. The additive can be an inorganic salt, or an organic compound that is available in the chemical reaction as a conjugate acid of a base (such as the base in its salt form) or in a free base form.

“Suppress,” as relates to an additive described herein, means to reduce or decrease the occurrence of a reaction pathway and/or formation of a product. This can be a complete or a partial reduction. Suppression can be compared to a control or to a standard level. The control can be a reaction condition involving the same reactants in the absence of the additive. Suppression can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

“Sterically hindered,” as relates to a nucleophile in an organic compound, refers to a nucleophile that has one or more substituents (such as two or more substituents) at a position α- or β- to the nucleophile in the organic compound. More specifically, “sterically hindered,” refers to a nucleophile that has one or more substituents (such as two or more substituents) at a position α- to the nucleophile in an organic compound. It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

“Substituted alkyl” refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF3, —CH₂—CF₃, —CCl₃); —CN; —NCOCOCH₂CH₂, —NCOCOCHCH; and —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom on the carbon backbone. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C═C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C═C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

“Aryl,” as used herein, refers to C₅-C₂₆-membered aromatic or fused aromatic ring systems. Examples of aromatic groups are benzene, naphthalene, anthracene, phenanthrene, chrysene, pyrene, corannulene, coronene, etc.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle” and “heterocyclyl” are used interchangeably, and refer to a cyclic radical attached via a ring carbon or nitrogen atom of a non-aromatic monocyclic or polycyclic ring containing 3-30 ring atoms, 3-20 ring atoms, 3-10 ring atoms, or 5-6 ring atoms, where each ring contains carbon and one to four heteroatoms each selected from the group consisting of non-peroxide oxygen, sulfur, and N (Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2, 3-b] tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1, 2, 5-thiadiazinyl. Heterocyclic groups can optionally be substituted with one or more substituents as defined above for alkyl and aryl.

The term “heteroaryl” refers to C₅-C₂₆-membered aromatic or fused aromatic ring systems, in which one or more carbon atoms on one or more aromatic ring structures have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1, 5, 2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1, 2, 3-oxadiazolyl, 1, 2, 4-oxadiazolyl, 1, 2, 5-oxadiazolyl, 1, 3, 4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1, 2, 3-thiadiazolyl, 1, 2, 4-thiadiazolyl, 1, 2, 5-thiadiazolyl, 1, 3, 4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.” The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

The term “polyaryl” refers to a chemical moiety that includes two or more fused aryl groups. When two or more fused heteroaryl groups are involved, the chemical moiety can be referred to as a “polyheteroaryl.”

The term “substituted polyaryl” refers to a polyaryl in which one or more of the aryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. When a polyheteroaryl is involved, the chemical moiety can be referred to as a “substituted polyheteroaryl.”

The term “cyclic ring” or “cyclic group” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, or a substituted or unsubstituted heterocyclyl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, and heterocyclyls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” or “alkoxy,” “aroxy” or “aryloxy,” generally describe compounds represented by the formula —OR^{v}, wherein R^{v} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by an oxygen as defined below. Accordingly, the substituent of an alkyl that renders that alkyl an ether is or resembles an alkoxyl, such as can be represented by one of —O-alkyl, —O-alkenyl, —O-alkynyl, —O-arakyl, —O-aryl, —O-heteroaryl, —O-polyaryl, —O-polyheteroaryl, —O-heterocyclyl, etc.

The term “substituted alkoxy” refers to an alkoxy group having one or more substituents replacing one or more hydrogen atoms on one or more carbons of the alkoxy backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, oxo, sulfate, sulfonate, sulfamoyl, sulfonamido, sulfonyl, heterocyclyl, alkylaryl, haloalkyl, —CN, aryl, heteroaryl, and combinations thereof.

The term “ether” as used herein is represented by the formula A²OA¹, where A² and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

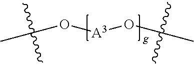

where A³ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art recognized and refers to a compound of the formula —OR^{v} wherein R^{v} is C₆H₅ (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “amino” as used herein includes the group

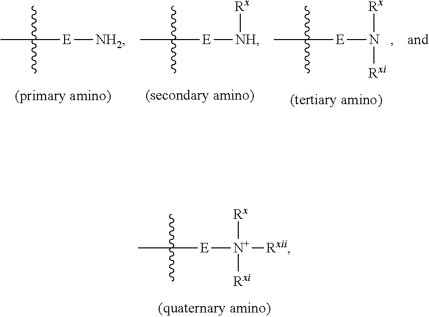

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure. It is understood by those of ordinary skill in the art, that the E groups listed above are divalent (e.g., methylene, ethane-1,2-diyl, ethene-1,2-diyl, 1, 4-phenylene, cyclohexane-1,2-diyl).

The terms “amide” or “amido” are used interchangeably, refer to both “unsubstituted amido” and “substituted amido” and are represented by the general formula:

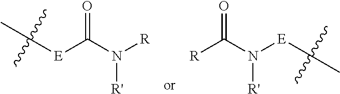

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘR′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed. It is understood by those of ordinary skill in the art, that the E groups listed above are divalent (e.g., methylene, ethane-1,2-diyl, ethene-1,2-diyl, 1, 4-phenylene, cyclohexane-1,2-diyl).

“Carbonyl,” as used herein, is art-recognized and includes such moieties as can be represented by the general formula:

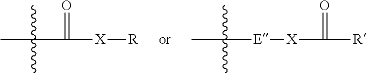

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is a substituted or unsubstituted alkyl, a substituted or unsubstituted alkenyl, a substituted or unsubstituted alkynyl, a substituted or unsubstituted aralkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. It is understood by those of ordinary skill in the art, that the E″ groups listed above are divalent (e.g., methylene, cthane-1,2-diyl, ethene-1,2-diyl, 1, 4-phenylene, cyclohexane-1,2-diyl). Where X is oxygen and R is defined as above, the moiety is also referred to as a carboxyl group. When X is oxygen and R is hydrogen, the formula represents a “carboxylic acid.” Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

The term “phosphanyl” is represented by the formula

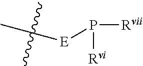

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1, 4-phenylene, cyclohexane-1,2-diyl).

The term “phosphonium” is represented by the formula

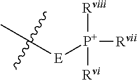

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi}, R^{vii}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi}, R^{vii}, and R^{viii} taken together with the P⁺ atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1, 4-phenylene, cyclohexane-1,2-diyl).

The term “phosphonyl” is represented by the formula

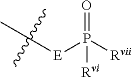

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, oxygen, alkoxy, aroxy, or substituted alkoxy or substituted aroxy, wherein, independently of E, R^{vi} and R^{vii} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1, 4-phenylene, cyclohexane-1,2-diyl).

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1, 4-phenylene, cyclohexane-1,2-diyl).

The term “sulfinyl” is represented by the formula

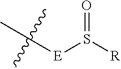

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂)ₘ−R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1, 4-phenylene, cyclohexane-1,2-diyl).

The term “sulfonyl” is represented by the formula

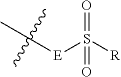

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1, 4-phenylenc, cyclohexane-1,2-diyl).

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, cthene-1,2-diyl, 1, 4-phenylene, cyclohexane-1,2-diyl).

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1, 4-phenylene, cyclohexane-1,2-diyl).

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R′″, R′″ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1, 4-phenylene, cyclohexane-1,2-diyl).

The term “sulfamoyl” refers to a sulfonamide or sulfonamide represented by the formula

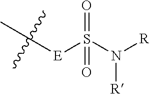

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1, 2-diyl, 1, 4-phenylene, cyclohexane-1,2-diyl).

The term “silyl group” as used herein is represented by the formula —SiRR′R, ″ where R, R′, and R″ can be, independently, a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, a phosphonyl, a sulfinyl, a thiol, an amido, an amino, an alkoxy, or an oxo, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted, independently, with the substituents described above in the definition of “substituted.”

The numerical ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and C₉, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

“oxo” refers to ═O.

The compounds and substituents can be substituted, independently, with the substituents described above in the definition of “substituted.”

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

### II. Conjugates, Methods of Making, and Reagents Therefor

Described herein are conjugates, such as bioconjugates, and methods of making the conjugates via intermolecular reactions between modular motifs. Traditionally, these intermolecular reactions have involved using a large excess of one modular motif to ensure the formation of the desired product. However, the use of the reactant in large excess (such as at least 10-fold equivalents) has limited the potential of this reaction and made it impractical for bioconjugation. It has now been discovered that the inclusion of an additive in the intermolecular reaction reduces or eliminates the need to utilize a large excess of one of the modular motifs. Hence, the product formed from at least three components, can be efficiently generated and achieve a stoichiometric reaction with comparable mole ratios of the reactants. Preferably, the reaction is performed in an aqueous buffer.

### A. Conjugates

In some forms, the disclosed conjugate contains a structure:

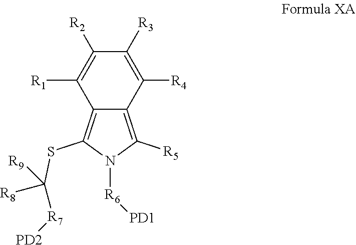

wherein:
R₁-R₄ are independently hydrogen, unsubstituted C₁-C₁₀ alkyl, substituted C₁-C ₁₀ alkyl, unsubstituted alkoxy, substituted alkoxy, unsubstituted aroxy, substituted aroxy, unsubstituted alkylthio, substituted alkylthio, unsubstituted carbonyl, substituted carbonyl, unsubstituted carboxyl, substituted carboxyl, unsubstituted ester, substituted ester, substituted amino, unsubstituted amino, substituted amide, unsubstituted amide, substituted aryl, unsubstituted aryl, substituted heteroaryl, unsubstituted heteroaryl, substituted polyaryl, unsubstituted polyaryl, substituted polyheteroaryl, a unsubstituted polyheteroaryl, substituted cycloalkyl, unsubstituted cycloalkyl, substituted cycloalkenyl, unsubstituted cycloalkenyl, substituted cycloalkynyl, unsubstituted cycloalkynyl, substituted C₁-C₂₀ heterocyclyl, unsubstituted C₁-C₂₀ heterocyclyl, or fused combination thereof,
R₅ is hydrogen, unsubstituted C₁-C₁₀ alkyl, substituted C₁-C₁₀ alkyl, unsubstituted C₁-C₁₀ alkenyl, substituted C₁-C₁₀ alkenyl, unsubstituted alkoxy, substituted alkoxy, unsubstituted aroxy, substituted aroxy, unsubstituted alkylthio, substituted alkylthio, unsubstituted carbonyl, substituted carbonyl, unsubstituted carboxyl, substituted carboxyl, unsubstituted ester, substituted ester, substituted amino, unsubstituted amino, substituted amide, unsubstituted amide, substituted aryl, unsubstituted aryl, substituted heteroaryl, unsubstituted heteroaryl, substituted polyaryl, unsubstituted polyaryl, substituted polyheteroaryl, a unsubstituted polyheteroaryl, substituted cycloalkyl, unsubstituted cycloalkyl, substituted cycloalkenyl, unsubstituted cycloalkenyl, substituted cycloalkynyl, unsubstituted cycloalkynyl, substituted C₁-C₂₀ heterocyclyl, unsubstituted C₁-C₂₀ heterocyclyl, or fused combination thereof,
R₆ and R⁷ are independently unsubstituted C₁-C₁₀ alkyl, substituted C₁-C₁₀ alkyl, substituted aryl, unsubstituted aryl, substituted heteroaryl, unsubstituted heteroaryl, substituted polyaryl, unsubstituted polyaryl, substituted polyheteroaryl, a unsubstituted polyheteroaryl, substituted cycloalkyl, unsubstituted cycloalkyl, substituted cycloalkenyl, unsubstituted cycloalkenyl, substituted cycloalkynyl, unsubstituted cycloalkynyl, substituted C₁-C₂₀ heterocyclyl, unsubstituted C₁-C₂₀ heterocyclyl, or fused combination thereof, with the proviso that R₆ and Ry are at least divalent, as valency permits,
R₈ and R₉ are independently hydrogen, unsubstituted C₁-C₁₀ alkyl, unsubstituted C₁-C₁₀ alkyl, with the proviso that at least one of R₈ and R₉ is not hydrogen, such as unsubstituted C₁-C₁₀ alkyl, or substituted C₁-C₁₀ alkyl, and
PD1 and PD2 independently contain a peptide, non-peptide therapeutic agent, non-peptide diagnostic agent, non-peptide prophylactic agent, or non-peptide targeting agent.

In some forms, the conjugate is as described above for Formula XA, except that R₁-R₄ are independently hydrogen, unsubstituted C₁-C₁₀ alkyl, or substituted C₁-C₁₀ alkyl. In some forms, the conjugate is as described above for Formula XA, except that R₁-R₄ are hydrogen. In some forms, the conjugate is as described above for Formula XA, except that at least one of R₁-R₄ independently contains a targeting agent, diagnostic agent, prophylactic agent, or a therapeutic agent. In some forms, the targeting agent contains a peptide.

In some forms, the conjugate is as described above for Formula XA, except that R₅ is hydrogen, unsubstituted C₁-C₁₀ alkyl, substituted C₁-C₁₀ alkyl, unsubstituted C₁-C₁₀ alkenyl, or substituted C₁-C₁₀ alkenyl.

In some forms, the conjugate is as described above for Formula XA, except that R₆ and R⁷ are independently unsubstituted C₁-C₁₀ alkyl, or substituted C₁-C₁₀ alkyl, with the proviso that R₆ and R⁷ are at least divalent, as valency permits. In some forms, the conjugate is as described above for Formula XA, except that R₆ and R⁷ independently contain a stimuli-responsive chemical moiety. In some forms, the conjugate is as described above for Formula XA, except that R₆ contains a stimuli-responsive chemical moiety. A stimuli-responsive moiety refers to a chemical group or bond that responds to an external stimulus. The stimuli-responsive chemical moiety can be cleaved by a stimulus selected from pH (such as change in pH), redox (such as change in redox potential), reactive oxygen species (ROS), enzyme (such as over-expression of an enzyme (e.g., a protease, esterase, etc.) due to a diseased state or disorder), ionic strength (such as a change in ionic strength), and hypoxia. Examples of stimuli-responsive chemical moieties that are responsive to ROS are described in Saravanakumar, et al., Adv. Sci. 2017, 4, 1600124, the contents of which are hereby incorporated by reference. In some forms, the stimuli-responsive chemical moiety independently contains a disulfide bond, an orthoester, a hydrazone, a hydrazide, a hydrazine, an imine, an oxime, an acetal group, a vinyl ether, a polyketal, a methyl maleate, an ester bond, an amide bond, a nitroaryl group (e.g., nitrobenzyl), a nitroheteroaryl (e.g, nitroimidazole), a quinone group, an azoaryl group (e.g., azophenyl), an azoheteroaryl group (e.g., azopyridinyl), peroxalate ester, aminoacrylate, alkyl thioether or selenide (e.g., monoselenide bond, diselenide bond, etc.), thioketal, peroxalate ester, or a dimethyl maleate. In some forms, the stimuli-responsive chemical moiety is independently a disulfide bond, an orthoester, a hydrazone, a hydrazide, a hydrazine, an imine, an oxime, a methyl maleate, or a dimethyl maleate. In some forms, the stimuli-responsive chemical moiety independently contains a disulfide bond, an orthoester, or a combination thereof. In some forms, the stimuli-responsive chemical moiety is a disulfide bond.

In some forms, the conjugate is as described above for Formula XA, except that R₈ and R₉ are independently unsubstituted C₁-C₁₀ alkyl or substituted C₁-C₁₀ alkyl.

In some forms, the conjugate is as described above for Formula XA, except that PD1 and PD2 contain a peptide. In some forms, the conjugate is as described above for Formula XA, except that one of PD1 and PD2 contains a peptide, and the other of PD1 and PD2 contains a non-peptide therapeutic agent, non-peptide diagnostic agent, non-peptide prophylactic agent, or non-peptide targeting agent. In some forms, the conjugate is as described above for Formula XA, except that one of PD1 and PD2 contains a peptide, and the other of PD1 and PD2 contains a non-peptide therapeutic agent. In some forms, the peptide in PD1 or PD2 is a targeting peptide. In some forms, the peptide in PD1, PD2, or both is an unprotected peptide. An unprotected peptide refers to a peptide that does not contain an amino acid-protecting group. Amino acid-protecting groups are reviewed in Isidro-Llobet, et al., Chem. Rev. 2009, 109 (6), 2455-2504, the contents of which are herein incorporated by reference. “Non-peptide,” as relates to therapeutic, prophylactic, diagnostic, or targeting agent, refers to these agents that do not contain two or more amino acid monomers bonded together by a peptide bond (—C(O)NH—).

### B. Methods of Making and Reagents Thereof

In some forms, methods of making the conjugates involve reacting intermolecularly a first reactant with a second reactant and a third reactant in the presence of an additive to form the conjugate. Preferably, the first reactant contains a structure:

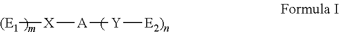

the second reactant contains a nucleophile denoted nu₁,
the third reactant contains a nucleophile denoted nu₂,
nu₁ and nu₂ are preferably selected from thiol groups, amine groups, and thiolate ions, preferably wherein nu₁ and nu₂ are not the same,
m and n are independently integers from 1 to 5, such as 1, 2, 3, 4, or 5, preferably m and n are 1,
A is a substituted aryl, an unsubstituted aryl, a substituted heteroaryl, an unsubstituted heteroaryl, a substituted polyaryl, an unsubstituted polyaryl, a substituted polyheteroaryl, an unsubstituted polyheteroaryl, a substituted cycloalkyl, an unsubstituted cycloalkyl, a substituted cycloalkenyl, an unsubstituted cycloalkenyl, a substituted cycloalkynyl, an unsubstituted cycloalkynyl, a substituted C₁-C₂₀ heterocyclyl, an unsubstituted C₁-C₂₀ heterocyclyl, or a fused combination thereof, such as substituted aryl fused with substituted heteroaryl, substituted aryl fused with unsubstituted heteroaryl, unsubstituted aryl fused with substituted heteroaryl, or substituted aryl fused with unsubstituted heteroaryl,
X and Y are independently absent, substituted alkyl, unsubstituted alkyl, substituted alkenyl, unsubstituted alkenyl, substituted alkynyl, unsubstituted alkynyl, unsubstituted alkoxy, substituted alkoxy, unsubstituted aroxy, substituted aroxy, unsubstituted alkylthio, substituted alkylthio, unsubstituted carbonyl, substituted carbonyl, unsubstituted carboxyl, substituted carboxyl, unsubstituted ester, substituted ester, unsubstituted ether, substituted ether, unsubstituted polyether, substituted polyether, substituted amino, unsubstituted amino, substituted amide, unsubstituted amide, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ Cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof, preferably X and Y are absent, and
E₁ and E₂ are electrophiles independently selected from substituted carbonyl or unsubstituted carbonyl, such as aldehydes, ketones, Michael acceptors; substituted carboxyl; or unsubstituted carboxyl. Preferably, the second reactant and the third reactant are not covalently linked prior to the reaction. Preferably, the first reactant, second reactant, and third reactant react with each other to form the conjugate containing a substituted 1-thio-isoindole moiety or an unsubstituted 1-thio-isoindole moiety. The phrase “intermolecularly” with respect to the reactants means that at least the second reactant and the third reactant are not covalently bonded to each other prior to the reaction.

In some forms, E₁ and E₂ are vicinal in the first reactant, i.e., they are directly or indirectly attached to adjacent atoms in a chemical ring or chain of the first reactant. Preferably, E₁ and E₂ are directly or indirectly attached to adjacent atoms in a chemical ring of the first reactant. More preferably, E₁ and E₂ are directly attached to vicinal atoms in a chemical ring or chain of the first reactant. Most preferably, E₁ and E₂ are directly attached to vicinal atoms in a chemical ring of the first reactant.

In some forms, the methods of making the conjugates are as described above, except that the additive contains a nucleophile. Preferably, the additive includes, but is not limited to, guanidines, ureas, thioureas, sodium bisulfite, or a combination thereof. Preferably, the additive suppresses an undesirable side reaction pathway in the reaction, with reference to a desired reaction pathway. In some forms, the additive associates (e.g., reacts) with the first reactant to form an adduct that suppresses the reaction between the third reactant (such as that containing an amine nucleophile) and the first reactant. In some forms, the additive suppresses a reaction pathway that leads to the formation of a two-component product involving only the first reactant and the third reactant. For instance, the addition of an additive, such as guanidine in its salt form (such as a guanidium, e.g., guanidium chloride) generates an OPA-guanidine adduct that can suppress the formation of imine-aldehyde that is a common intermediate in the non-selective formation of two-component and three-component products.

In some forms, the methods of making the conjugates are as described above, except that the concentration of the additive is between about 0.5 M and about 10 M, between about 1 M and about 8 M, or between about 3 M and about 8 M, such as 1 M, 2 M, 3 M, 4 M, 5 M, 6 M, 7 M, 8 M, 9 M, or 10 M, preferably 1 M, 3 M, 6 M, and 8 M. As described herein, this concentration preferably refers to the concentration of the additive prior to being added to a solvent system, such as a buffer, in which the reaction will proceed.

In some forms, the methods of making the conjugates are as described above, except that the mole ratio of the first reactant to the second reactant is maintained between 1:8 to 8:1. Preferably, the mole ratio of the first reactant to the second reactant is maintained between 1:5 to 5:1, more preferably between 1:4 to 4:1, or between 1:3 to 3:1, such as 1:1.2, 1:1, or 2:1.2.

In some forms, the methods of making the conjugates are as described above, except that the mole ratio of the second reactant to the third reactant is between 1:4 to 4:1. Preferably, the mole ratio of the second reactant to the third reactant is between 1:4 to 4:1, or between 1:3 to 3:1, such as 1:1, 1:1.5, 3:1, or 2:1.

In some forms, the methods of making the conjugates are as described above, except that the second reactant and the third reactant are independently therapeutic agents, prophylactic agents, diagnostic agents, or targeting agents. In some forms, the second reactant and the third reactant are independently linear peptides, cyclic peptides, non-polymeric small molecule drugs (such as those having a molecular weight between 100 Da and 2, 500 Da), fluorophores, chemiluminescent compounds, or a combination thereof.

In some forms, the methods of making the conjugates are as described above, except that the second reactant contains a thiol group that reacts with the first reactant. In some forms, the second reactant contains a sterically hindered thiol group that reacts with the first reactant.

In some forms, the methods of making the conjugates are as described above, except that the third reactant contains an amine group that reacts with the first reactant.

In some forms, the methods of making the conjugates are as described above, except that the first reactant has a structure:

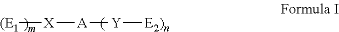

wherein:
A is a substituted aryl, an unsubstituted aryl, a substituted heteroaryl, an unsubstituted heteroaryl, a substituted polyaryl, an unsubstituted polyaryl, a substituted polyheteroaryl, an unsubstituted polyheteroaryl, a substituted C₁-C₂₀ heterocyclyl, an unsubstituted C₁-C₂₀ heterocyclyl, or a fused combination thereof. Preferably, E₁ and E₂ are vicinal in the first reactant, i.e., they are attached to adjacent atoms in a chemical ring or chain. Preferably, E₁ and E₂ are attached to adjacent atoms in a chemical ring of the first reactant.

In some forms, the methods of making the conjugates are as described above, except that for Formula I (encompassing the first reactant), A is a substituted aryl or an unsubstituted aryl.

In some forms, the methods of making the conjugates are as described above, except that Formula I has a structure:

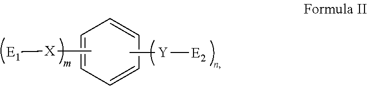

Preferably, in Formula II, E₁ and E₂ are vicinal in the aromatic ring.

In some forms, the methods of making the conjugates are as described above, except that for Formula I and Formula II, m and n are 1.

In some forms, the methods of making the conjugates are as described above, except that Formula I or Formula II has a structure:

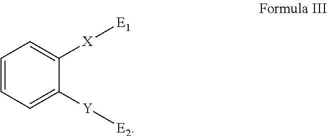

In some forms, the methods of making the conjugates are as described above, except that Formula I, Formula II, or Formula III has a structure:

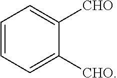

In some forms, the methods of making the conjugates are as described above, except that the method involves mixing the additive with a suitable buffer, such as phosphate-buffered saline (PBS), to form a solution prior to adding the first, second, and third reactants. Preferably, after adding the additive to the buffer, the pH of the resultant solution is adjusted to a desired value. In some forms, the pH of the resultant solution is adjusted to about 7.4. This pH adjustment can be achieved using a solution of 1 M sodium hydroxide.

In some forms, the methods of making the conjugates are as described above, except that the method involves adding the first reactant and the second reactant in a solution containing the additive and a suitable buffer, prior to adding the third reactant. It is preferable that the second reactant is added first, to suppress (preferably completely) the undesired two-component product formed exclusively from the first reactant and the third reactant.

In some forms, the methods of making the conjugates are as described above, except that the buffer has a pH between about 7.0 and about 7.5, such as about 7.4.

In some forms, the methods of making the conjugates are as described above, except that the reaction is performed at a temperature, between about 23° C. and about 40° C. In some forms, the reaction is performed at room temperature, such as between about 23° C. and about 28° C. In some forms, the reaction is performed between about 30° C. and about 40° C., such as at about 37° C. It is to be understood that the temperature is selected such that the relevant reactant (such as a protein, peptide, etc.) is not denatured during the reaction.

In some forms, the methods of making the conjugates are as described above, except that the reaction has a concentration between about 0.1 mM and about 7.5 mM, between about 0.1 mM and about 5 mM, between about 0.1 mM and about 2.5 mM, between about 0.1 mM and about 2 mM, between about 0.5 mM and about 2.5 mM, or between about 0.5 mM and about 2 mM.

In some forms, the methods of making the conjugates are as described above, except that the reaction involves:
(i) adding the additive in a suitable buffer, such as PBS, to form a solution, dispersion, or suspension,
(ii) adding the first reactant and the second reactant in the solution, dispersion, or suspension of step (i) to form another solution, dispersion, or suspension, and
(iii) adding the third reactant to the solution of step (ii) to form another solution, dispersion, or suspension. In some forms, the solution, dispersion, or suspension of step (iii) is maintained in an air atmosphere and at a temperature between about 23° C. and about 40° C. In some forms, the solution, dispersion, or suspension of step (iii) is maintained at a room temperature, such as between about 23° C. and about 28° C. In some forms, the solution, dispersion, or suspension of step (iii) is maintained at a temperature between about 30° C. and about 40° C., such as at about 37° C. It is to be understood that the temperature is selected such that the relevant reactant (such as a protein, peptide, etc.) is not denatured during the reaction.

The disclosed methods and conjugates can be further understood through the following numbered paragraphs.

1. A method of making a conjugate, the method involving:
reacting intermolecularly a first reactant, a second reactant, and a third reactant in the presence of an additive to form the conjugate, preferably wherein the additive suppresses a product formed by a reaction between only the first reactant and the third reactant,
wherein:
the first reactant contains a structure:

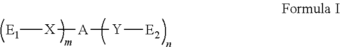

the second reactant contains a nucleophile denoted nu₁,
the third reactant contains a nucleophile denoted nu₂,
preferably, the second reactant and the third reactant are not covalently linked prior to the reaction,
nu₁ and nu₂ are preferably selected from thiol groups, amine groups, and thiolate ions, preferably wherein nu₁ and nu₂ are not the same,
m and n are independently integers from 1 to 5, such as 1, 2, 3, 4, or 5, preferably, m and n are 1,
A is a substituted aryl, an unsubstituted aryl, a substituted heteroaryl, an unsubstituted heteroaryl, a substituted polyaryl, an unsubstituted polyaryl, a substituted polyheteroaryl, an unsubstituted polyheteroaryl, a substituted cycloalkyl, an unsubstituted cycloalkyl, a substituted cycloalkenyl, an unsubstituted cycloalkenyl, a substituted cycloalkynyl, an unsubstituted cycloalkynyl, a substituted C₁-C₂₀ heterocyclyl, an unsubstituted C₁-C₂₀ heterocyclyl, or a fused combination thereof, such as substituted aryl fused with substituted heteroaryl, substituted aryl fused with unsubstituted heteroaryl, unsubstituted aryl fused with substituted heteroaryl, or substituted aryl fused with unsubstituted heteroaryl,
X and Y are independently absent, substituted alkyl, unsubstituted alkyl, substituted alkenyl, unsubstituted alkenyl, substituted alkynyl, unsubstituted alkynyl, unsubstituted alkoxy, substituted alkoxy, unsubstituted aroxy, substituted aroxy, unsubstituted alkylthio, substituted alkylthio, unsubstituted carbonyl, substituted carbonyl, unsubstituted carboxyl, substituted carboxyl, unsubstituted ester, substituted ester, unsubstituted ether, substituted ether, unsubstituted polyether, substituted polyether, substituted amino, unsubstituted amino, substituted amide, unsubstituted amide, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ cycloalkynyl, or fused combinations thereof, and
E₁ and E₂ are electrophiles independently selected from substituted carbonyl or unsubstituted carbonyl, such as aldehydes, ketones, Michael acceptors; substituted carboxyl; or unsubstituted carboxyl.

2. The method of paragraph 1, wherein the additive contains a nucleophile, preferably selected from guanidines, ureas, thioureas, sodium bisulfite, and combinations thereof.

3. The method of paragraph 1 or 2, wherein the concentration of the additive is between about 0.5 M and about 10 M, between about 1 M and about 8 M, or between about 3 M and about 8 M, such as 1 M, 2 M, 3 M, 4 M, 5 M, 6 M, 7 M, 8 M, 9 M, or 10 M, preferably 1 M, 3 M, 6 M, and 8 M.

4. The method of any one of paragraphs 1 to 3, wherein the mole ratio of the first reactant to the second reactant is between 1:8 to 8:1, between 1:5 to 5:1, between 1:4 to 4:1, or between 1:3 to 3:1, such as 1:1.2, 1:1, or 2:1.2.

5. The method of any one of paragraphs 1 to 4, wherein the mole ratio of the second reactant to the third reactant is between 1:4 to 4:1 or between 1:3 to 3:1, such as 1:1, 1:1.5, 3:1, or 2:1.

6. The method of any one of paragraphs 1 to 5, wherein the second reactant and the third reactant are independently therapeutic agents, prophylactic agents, diagnostic agents, or targeting agents.

7. The method of any one of paragraphs 1 to 6, wherein the second reactant and the third reactant are independently linear peptides, cyclic peptides, non-polymeric small molecule drugs (such as those having a molecular weight between 100 Da and 2, 500 Da), fluorophores, chemiluminescent compounds, or a combination thereof.

8. The method of any one of paragraphs 1 to 7, wherein the second reactant contains a thiol group that reacts with the first reactant.

9. The method of any one of paragraphs 1 to 8, wherein the second reactant contains a sterically hindered thiol group that reacts with the first reactant.

10. The method of any one of claims 1 to 9, wherein the third reactant contains an amine group that reacts with the first reactant.

11. The method of any one of claims 1 to 10, wherein reacting the first reactant, second reactant, and third reactant forms the conjugate containing a substituted 1-thio-isoindole moiety or an unsubstituted 1-thio-isoindole moiety.

12. The method of any one of claims 1 to 11, wherein the first reactant has a structure:

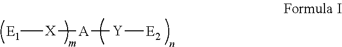

wherein:
A is a substituted aryl, an unsubstituted aryl, a substituted heteroaryl, an unsubstituted heteroaryl, a substituted polyaryl, an unsubstituted polyaryl, a substituted polyheteroaryl, an unsubstituted polyheteroaryl, a substituted C₁-C₂₀ heterocyclyl, an unsubstituted C₁-C₂₀ heterocyclyl, or a fused combination thereof.

13. The method of any one of paragraphs 1 to 12, wherein for Formula I, A is a substituted aryl or an unsubstituted aryl.

14. The method of any one of paragraphs 1 to 13, wherein the first reactant has a structure:

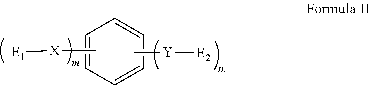

15. The method of any one of paragraphs 1 to 14, wherein m and n are 1.

16. The method of any one of paragraphs 1 to 15, wherein the first reactant has a structure:

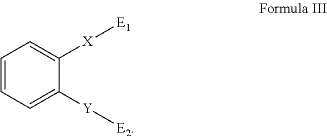

17. The method of any one of paragraphs 1 to 16, wherein the first reactant has a structure:

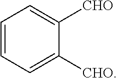

18. The method of any one of paragraphs 1 to 17, wherein the reaction is performed in an aqueous buffer.

19. The method of any one of paragraphs 1 to 18, involving mixing the additive with a suitable buffer, such as phosphate-buffered saline (PBS), to form a solution prior to adding the first, second, and third reactants.

20. The method of any one of paragraphs 1 to 19, involving adding the first reactant and the second reactant in a solution containing the additive and a suitable buffer, prior to adding the third reactant.

21. The method of paragraph 19 or 20, wherein the buffer has a pH between about 7.0 and about 7.5.

22. The method of any one of paragraphs 1 to 21, wherein the reaction is performed at room temperature, such as between about 23° C. and about 37° C., or about 23° C. and about 28° C.

23. The method of any one of paragraphs 1 to 22, wherein the reaction has a concentration between about 0.1 mM and about 7.5 mM, between about 0.1 mM and about 5 mM, between about 0.1 mM and about 2.5 mM, between about 0.1 mM and about 2 mM, between about 0.5 mM and about 2.5 mM, or between about 0.5 mM and about 2 mM.

24. The method of any one of paragraphs 1 to 23, involving:
(i) adding the additive in a suitable buffer, such as PBS, to form a solution, dispersion, or suspension,
(ii) adding the first reactant and the second reactant in the solution, dispersion, or suspension of step (i) to form another solution, dispersion, or suspension, and
(iii) adding the third reactant to the solution of step (ii) to form another solution, dispersion, or suspension.

25. The method of paragraph 24, wherein the solution, dispersion, or suspension of step (iii) is maintained in an air atmosphere and at a temperature between about 23° C. and about 40° C., between about 23° C. and about 28° C., or between about 30° C. and about 40° C., to form the conjugate.

26. A conjugate having a structure:

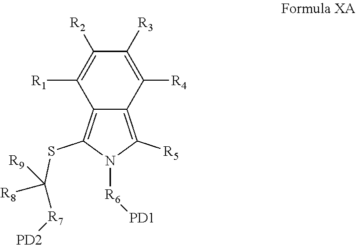

wherein:
R₁-R₄ are independently hydrogen, unsubstituted C₁-C₁₀ alkyl, substituted C₁-C₁₀ alkyl, unsubstituted alkoxy, substituted alkoxy, unsubstituted aroxy, substituted aroxy, unsubstituted alkylthio, substituted alkylthio, unsubstituted carbonyl, substituted carbonyl, unsubstituted carboxyl, substituted carboxyl, unsubstituted ester, substituted ester, substituted amino, unsubstituted amino, substituted amide, unsubstituted amide, substituted aryl, unsubstituted aryl, substituted heteroaryl, unsubstituted heteroaryl, substituted polyaryl, unsubstituted polyaryl, substituted polyheteroaryl, a unsubstituted polyheteroaryl, substituted cycloalkyl, unsubstituted cycloalkyl, substituted cycloalkenyl, unsubstituted cycloalkenyl, substituted cycloalkynyl, unsubstituted cycloalkynyl, substituted C₁-C₂₀ heterocyclyl, unsubstituted C₁-C₂₀ heterocyclyl, or fused combination thereof,
R₅ is hydrogen, unsubstituted C₁-C₁₀ alkyl, substituted C₁-C₁₀ alkyl, unsubstituted C₁-C₁₀ alkenyl, substituted C₁-C₁₀ alkenyl, unsubstituted alkoxy, substituted alkoxy, unsubstituted aroxy, substituted aroxy, unsubstituted alkylthio, substituted alkylthio, unsubstituted carbonyl, substituted carbonyl, unsubstituted carboxyl, substituted carboxyl, unsubstituted ester, substituted ester, substituted amino, unsubstituted amino, substituted amide, unsubstituted amide, substituted aryl, unsubstituted aryl, substituted heteroaryl, unsubstituted heteroaryl, substituted polyaryl, unsubstituted polyaryl, substituted polyheteroaryl, a unsubstituted polyheteroaryl, substituted cycloalkyl, unsubstituted cycloalkyl, substituted cycloalkenyl, unsubstituted cycloalkenyl, substituted cycloalkynyl, unsubstituted cycloalkynyl, substituted C₁-C₂₀ heterocyclyl, unsubstituted C₁-C₂₀ heterocyclyl, or fused combination thereof,
R₆ and R⁷ are independently unsubstituted C₁-C₁₀ alkyl, substituted C₁-C₁₀ alkyl, substituted aryl, unsubstituted aryl, substituted heteroaryl, unsubstituted heteroaryl, substituted polyaryl, unsubstituted polyaryl, substituted polyheteroaryl, a unsubstituted polyheteroaryl, substituted cycloalkyl, unsubstituted cycloalkyl, substituted cycloalkenyl, unsubstituted cycloalkenyl, substituted cycloalkynyl, unsubstituted cycloalkynyl, substituted C₁-C₂₀ heterocyclyl, unsubstituted C₁-C₂₀ heterocyclyl, or fused combination thereof, with the proviso that R₆ and R⁷ are at least divalent, as valency permits,
R₈ and R₉ are independently hydrogen, unsubstituted C₁-C₁₀ alkyl, substituted C₁-C₁₀ alkyl, with the proviso that at least one of R₈ and R₉ is not hydrogen, such as unsubstituted C₁-C₁₀ alkyl, or substituted C₁-C₁₀ alkyl, and
PD1 and PD2 independently contain a peptide, non-peptide therapeutic agent, non-peptide diagnostic agent, non-peptide prophylactic agent, or non-peptide targeting agent.

27. The conjugate of paragraph 26, wherein R¹-R₄ are independently hydrogen, unsubstituted C₁-C₁₀ alkyl, or substituted C₁-C₁₀ alkyl.

28. The conjugate of paragraph 26 or 27, wherein R¹-R₄ are hydrogen.

29. The conjugate of paragraph 26 or 27, wherein at least one of R₁-R₄ independently contains a targeting agent, diagnostic agent, prophylactic agent, or a therapeutic agent, preferably wherein the targeting agent contains a peptide.

30. The conjugate of any one of paragraphs 26 to 29, wherein R⁵ is hydrogen, unsubstituted C₁-C₁₀ alkyl, substituted C₁-C₁₀ alkyl, unsubstituted C₁-C₁₀ alkenyl, or substituted C₁-C₁₀ alkenyl.

31. The conjugate of any one of paragraphs 26 to 30, wherein R⁶ and R⁷ are independently unsubstituted C₁-C₁₀ alkyl, or substituted C₁-C₁₀ alkyl, with the proviso that R₆ and R⁷ are at least divalent, as valency permits.

32. The conjugate of any one of paragraphs 26 to 31, wherein R⁶ and R⁷ independently contain a stimuli-responsive chemical moiety, preferably wherein R⁶ contains a stimuli-responsive chemical moiety.

33. The conjugate of any one of paragraphs 26 to 32, wherein R₈ and R₉ are independently unsubstituted C₁-C₁₀ alkyl or substituted C₁-C₁₀ alkyl.

34. The conjugate of any one of paragraphs 26 to 33, wherein PD1 and PD2 contain a peptide, preferably an unprotected peptide.

35. The conjugate of any one of paragraphs 26 to 34, wherein one of PD1 and PD2 contains a peptide, preferably an unprotected peptide, and the other of PD1 and PD2 contains a non-peptide therapeutic agent, non-peptide diagnostic agent, non-peptide prophylactic agent, or non-peptide targeting agent.

36. The conjugate of any one of paragraphs 26 to 35, wherein one of PD1 and PD2 contains a peptide, preferably an unprotected peptide, and the other of PD1 and PD2 contains a non-peptide therapeutic agent.

### EXAMPLES

### Example 1: Efficient Synthesis Conjugates Containing 1-Thio-Isoindole Cores Using of Three-Component Reactions

### Materials and Methods

All commercially available amino acids and coupling reagents (purchased from Aldrich and CS Bio) were used without further purification. All solvents in reagent grade (RCI) or HPLC grade (DUKSAN) were used without purification. Anhydrous dichloromethane (DCM) was freshly distilled from calcium hydride (CaH₂) before use. Analytical HPLC was performed on a Waters system equipped with a photodiode array detector (Waters 2996), using a Vydac 218TPTM C₁₈ column (5 μm, 4.6×250 mm) at a flow rate of 0.6 mL/min. Waters UPLC H-class system equipped with an ACQUITY UPLC photodiode array detector and a Waters SQ Detector 2 mass spectrometer using a Waters ACQUITY BEH C₁₈ column (1.7 μm, 130 Å, 2.1×50 mm) at a flow rate of 0.4 mL/min. Preparative HPLC was performed on a Waters system, using a Vydac 218TPTM C₁₈ column (10 μm, 22×250 mm) at a flow rate of 10 mL/min or a Vydac 218TPTM C₁₈ column (10 μm, 30×250 mm) at a flow rate of 20 mL/min. Mobile phases of HPLC used are as followed: Solvent A: 0.1% TFA (v/v) in acetonitrile; Solvent B: 0.1% TFA (v/v) in water. Mass analysis was performed with a Waters 3100 mass spectrometer.

### Solid Phase Peptide Synthesis

Synthesis was performed manually on rink amide resin (CS Bio) under the standard Fmoc protocol. Removal of Fmoc protecting group was performed using a mixture of 20/80 (v/v) of piperidine/DMF for 20 min. Coupling was performed using Fmoc-Amino acids (4.0 equiv.), HATU (4.0 equiv.) and DIPEA (8.0 equiv.) in DMF for 1 hour at room temperature. Acetylation of the peptide N-terminus was performed using AcOH (8.0 equiv.) and DIPEA (16.0 equiv.) in DMF for 1 h at room temperature. Upon completion of the synthesis, 95:2.5:2.5(%) of TFA:TIPS:Water (v:v:v) were used to perform global deprotection. The peptides were then precipitated in cold diethyl ether and purified by preparative RP-HPLC.

### General Procedure for the OPA Three-Component Reactions

Preparation of the stock solution: Stock solution of OPA (1 mg/100 μL DMSO) was freshly prepared before use. For the OPA derivatives, OPA-fluorescein and OPA-peptide, the stock solution was prepared at 1 mg/10 μL DMSO. For the peptide-drug conjugates, components consisting of DM1 were freshly dissolved in 3% ACN, MeOH and DMSO (of the total reaction volume) respectively before use.

Preparation of the PBS buffer containing guanidine: Guanidine hydrochloride (1 M to 8 M) was added to the PBS buffer (137 mM NaCl, 2.7 mM KCl, 10 mM Na₂HPO₄, and 1.8 mM KH₂PO₄). The pH of the solution was adjusted to 7.4 by the addition of 1 M NaOH solution.

### Synthesis of the Peptide-Peptide Conjugates

Condition [A]: Thiol-containing peptide (1.0 equiv.) and OPA (1.2 equiv.) were first dissolved in PBS buffer pH 7.4, at a concentration of 2 mM. The amine-containing peptide (1.0 equiv.) was then added and reacted in an air atmosphere for 1 h at room temperature (rt). The crude reaction mixture was then purified by preparative reverse-phase HPLC.

Condition [B]: Thiol-containing peptide (1.0 equiv.) and OPA (1.2 equiv.) were first dissolved in PBS buffer pH 7.4 with guanidine at a concentration of 2 mM. (The concentration of guanidine applied for the peptide-peptide conjugate models was determined on a case-by-case basis. It ranged from 3 M to 8 M, as specified below per entry.) The amine-containing peptide (1.0 equiv.) was then added and reacted in an air atmosphere for 1 h at rt. The crude reaction mixture was then purified by preparative reverse-phase HPLC.

### Synthesis of the Peptide-Drug Conjugates

Condition [C]: DM1 (1.0 equiv.) and OPA (1.2 equiv.) were first dissolved in PBS buffer pH 7.4 with 1 M guanidine, at a final concentration of 0.5 mM. The c [RGDfK] or c [RGEfK] peptide (1.5 equiv.) was then added and reacted in an air atmosphere for 1 h at rt. The crude reaction mixture was then purified by preparative reverse-phase HPLC.

Condition [D]: DM1 (1.0 equiv.) and OPA (1.2 equiv.) were first dissolved in PBS buffer pH 7.4 with 1 M guanidine, at a final concentration of 0.5 mM. The c [RGDfK] or c [RGEfK] peptide (1.5 equiv.) was then added and reacted in an air atmosphere for 1 h at rt. Afterwards, guanidine was added to the crude reaction mixture to achieve an 8 M guanidine buffer, followed by the addition of DMAC (2.0 equiv.) and reacted for another 1 h. The crude reaction mixture was purified by preparative reverse-phase HPLC.

Condition [E]: c [RGDfPen] or c [RGEfPen] peptide (1.0 equiv.) and OPA (1.2 equiv.) were first dissolved in PBS buffer pH 7.4 with 6 M guanidine, at a concentration of 5 mM. H₂N—CH₂CH₂—S—S-DM1 (1.0 equiv.) was then added and reacted in an air atmosphere for 3 h at rt. The crude reaction mixture was then purified by preparative reverse-phase HPLC.

Condition [F]: c [RGDf (K-Pen)] (3.0 equiv.) and OPA-fluorescein (3.0 equiv.) were first dissolved in PBS buffer pH 7.4 with 6 M guanidine, at a concentration of 1 mM. H₂N—CH₂CH₂—δ-S-DM1 (1.0 equiv.) was then added. The reaction was carried out at 37° C. in an air atmosphere for 2 h. The crude reaction mixture was then purified by preparative reverse-phase HPLC.

Condition [G]: c [RGDf (K-Pen)] (2.0 equiv.) and OPA-peptide (1.2 equiv.) were first dissolved in PBS buffer pH 7.4 with 6 M guanidine, at a concentration of 1 mM. H₂N—CH₂CH₂—S—S-DM1 (1.0 equiv.) was then added. The reaction was carried out at rt in an air atmosphere for 2 h. The crude reaction mixture was then purified by preparative reverse-phase HPLC.

### Synthesis of PPC-2

The thiol-containing fragment (2.02 mg, 0.0016 mmol, 1.0 equiv.) and OPA (0.26 mg, 26 μL stock solution in DMSO, 0.0019 mmol, 1.2 equiv.) were first dissolved in PBS buffer pH 7.4 with 3 M guanidine, at a concentration of 2 mM (800 μL). The amine-containing fragment (0.97 mg, 0.0016 mmol, 1.0 equiv.) was then added and reacted in an air atmosphere for 1 h at rt. The crude reaction mixture was then purified by preparative reverse-phase HPLC (H₂O and ACN with 0.1% TFA as cluents) to give PPC-2 as a white powder after lyophilization (1.57 mg, 50% yield). The peptide-peptide conjugates were constructed according to the similar procedure with details listed in condition [B] while peptide-drug conjugates were constructed according to the similar procedure with details listed in conditions [C] to [G].

### In Vitro Cytotoxicity Assay

Cells were plated in 96-well microplates at a density of 5×10⁴ cells/well for 24 h for attachment, 90 μl of DMEM with 10% FBS and 100 U/mL P/Swas added to each well. PDCs 1-6 were first dissolved in DMSO respectively, at a concentration of 10 mM, followed by serial dilution in culture medium to achieve different concentrations as specified. For U87-MG cells, the concentration of compounds administered ranged from 1.37 to 27000 nM. While for HEK-293T cells, the concentration of compounds administered ranged from 0.03 to 1000 nM. The cells treated with the compounds were then incubated for 48 h. 10 μl of CCK-8 solution was then added to each well and incubated for 30 min. Absorbance was acquired at 450 nm using a spectrophotometer (Synergy HTX, Biotek). Three experiments were performed separately in triplicates and the data was processed using the GraphPad Prism (GraphPad Software, La Jolla, CA).

### Fluorescence Confocal Imaging Analysis

U87-MG cells were seeded at 2×10⁵ cells/well in the 35 mm glass-bottom dish for attachment. 1 mL of DMEM with 10% FBS and 100 U/mL P/Swas added to the dish. After 24 h incubation, the medium was discarded. 1 mL of DMEM with 10% FBS and 100 U/mL P/Scontaining PDC-7 (final concentration: 10 μM/mL) and DAPI (final concentration: 1 μg/mL) was freshly prepared. The resultant solution was added to the cells and incubated for 15 min. Afterwards, the supernatant was discarded and the cells were washed with PBS for three times. The cells were replenished with PBS and subjected to fluorescence confocal imaging (Leica DMI3000b). The images were captured and processed via Leica Application Suit X at ×40 oil objective (Leica).

### Results

A series of compounds that could react with the aldehyde groups and suppress the side reaction pathway were screened, including guanidine, urea, thiourea and sodium bisulfite (Table 1). The additives were dissolved in the buffer and adjusted to the desired pH before use. The thiol-carrying peptide Ac-QSQQTFSNLWRLLCQN-NH₂ (SEQ ID NO: 1) (1.0 equiv.) and OPA (1.2 equiv.) were then dissolved in the indicated buffer, followed by the addition of the amine-carrying peptide Ac-QSQQTFKNLWRLLPQN-NH₂ (SEQ ID NO: 2) (1.0 equiv.). The reactions were monitored by LCMS. Guanidine appeared to show the best results amongst the additives and resulted in excellent selectivity towards the desired conjugates.

TABLE 1
Conditions for Screening Potential Compounds

| | | | Conc. | | Reaction | | Conversion^{b} |
|---|---|---|---|---|---|---|---|
| Entry | Buffer | Additive | (mM) | Temperature | Time^{a} | | (Selectivity^{c}) |
| 1 | PBS Buffer | — | 0.5 | RT | 1 | hour | >99% (40%) |
| | pH 7.4 | | | | | | |
| 2 | PBS Buffer | 6M guanidine | 0.5 | RT | >7 | hours | 77% (>99%) |
| | pH 7.4 | | | | | | |
| 3 | PBS Buffer | 6M urea | 0.5 | RT | 1 | hour | 94% (68%) |
| | pH 7.4 | | | | | | |
| 4 | PBS Buffer | 4M urea + 2M | 0.5 | RT | 1 | hour | 89% (61%) |
| | pH 7.4 | thiourea | | | | | |
| 5 | PBS Buffer | 6M urea + 6M | 0.5 | RT | >7 | hours | 28% (>99%) |
| | pH 7.4 | guanidine | | | | | |
| 6 | PBS Buffer | Sodium | 0.5 | RT | >7 | hours | 54% (>99%) |
| | pH 7.4 | bisulfite | | | | | |
| | | (2equiv.) | | | | | |
| 7 | PBS Buffer | 6M guanidine | 0.5 | 40° C. | >3 | hours | 31% (>99%) |
| | pH 7.4 | | | | | | |
| 8 | Borate | 6M guanidine | 0.5 | RT | >3 | hours | 45% (>99%) |
| | buffer | | | | | | |
| | pH 8.5 | | | | | | |
| 9 | PBS buffer | 1M guanidine | 0.5 | RT | 2 | hours | 86% (87%) |
| | pH 7.4 | | | | | | |
| 10 | PBS Buffer | 3M guanidine | 0.5 | RT | >2 | hours | 80% (98%) |
| | pH 7.4 | | | | | | |
| 11 | PBS Buffer | 3M guanidine | 2 | RT | 1 | hour | 86% (93%) |
| | pH 7.4 | | | | | | |

^{a}Time required for the reaction to complete.
^{b}Percentage of conversion calculated based on the LCMS analysis of the crude reaction mixture.
Majority of the by-products were unreacted starting materials unless specified.
^{c}Selectivity towards the three-component product.

Additional experiments involving this OPA-amine-thiol three-component reaction through the synthesis of peptide-peptide conjugates (PPCs) were performed. To test the chemoselectivity of the method, a series of thiol-containing unprotected peptides (with a Cys side chain) and amine-containing unprotected peptides (with a Lys side chain or N-terminus) consisting of all proteinogenic amino acids were prepared. The thiol fragment (1.0 equiv.) and OPA (1.2 equiv.) were first added to the PBS buffer containing guanidine, followed by the amine fragment (1.0 equiv.). The reaction was carried out at a concentration of 2 mM and reacted in an air atmosphere for 1 hour at room temperature (RT). Control experiments were conducted under identical conditions, but no guanidine was added.

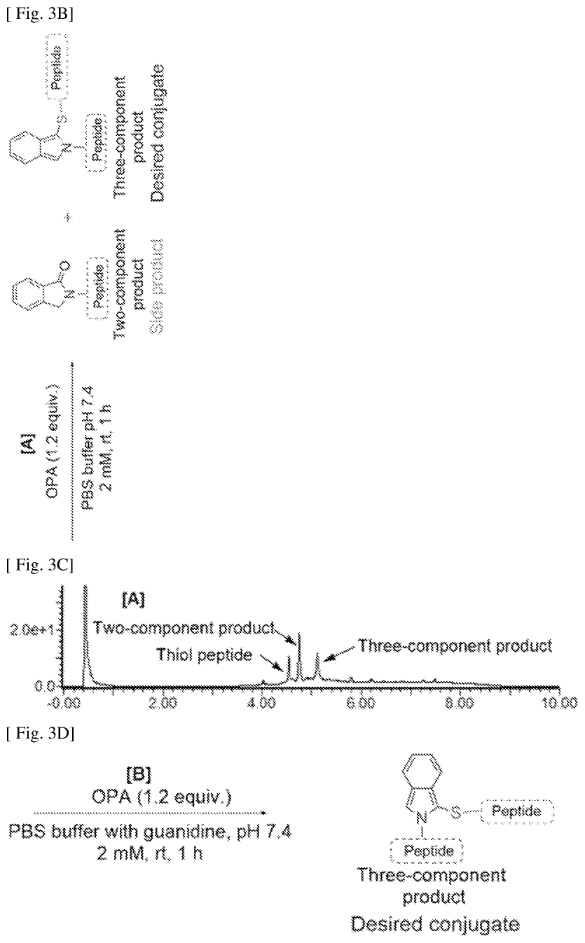

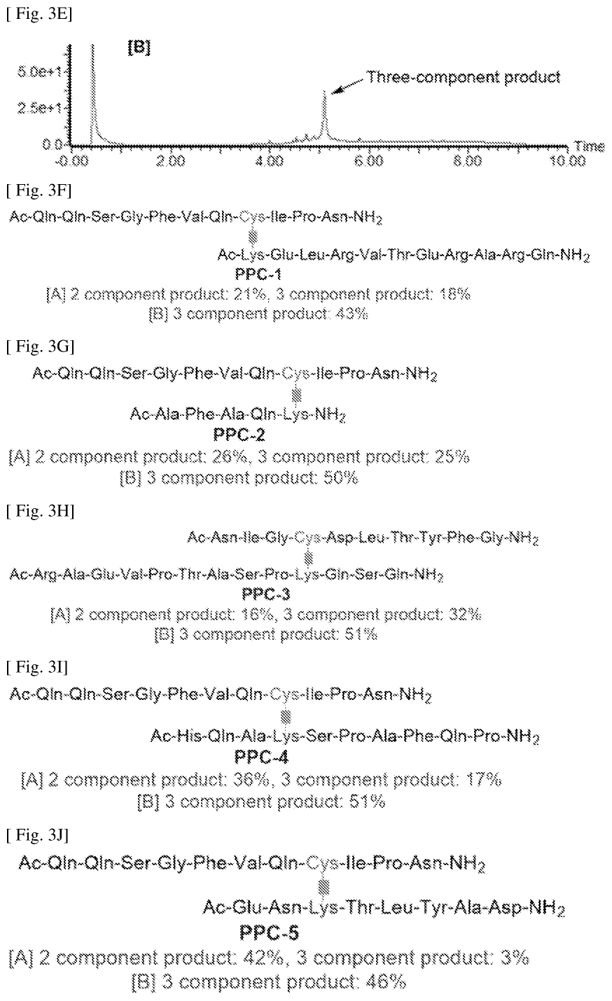

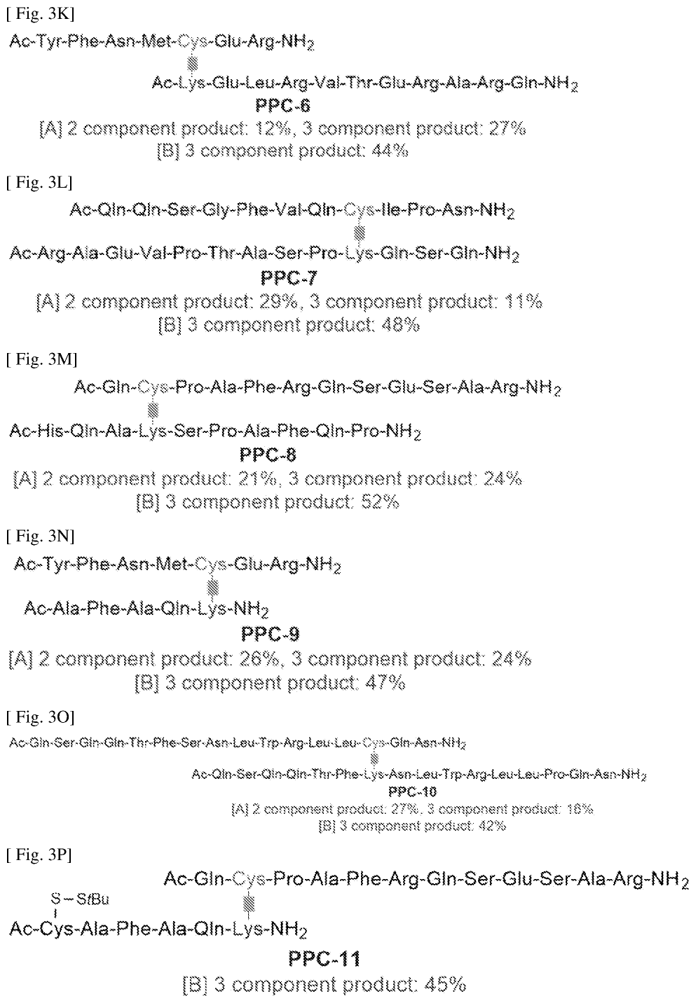

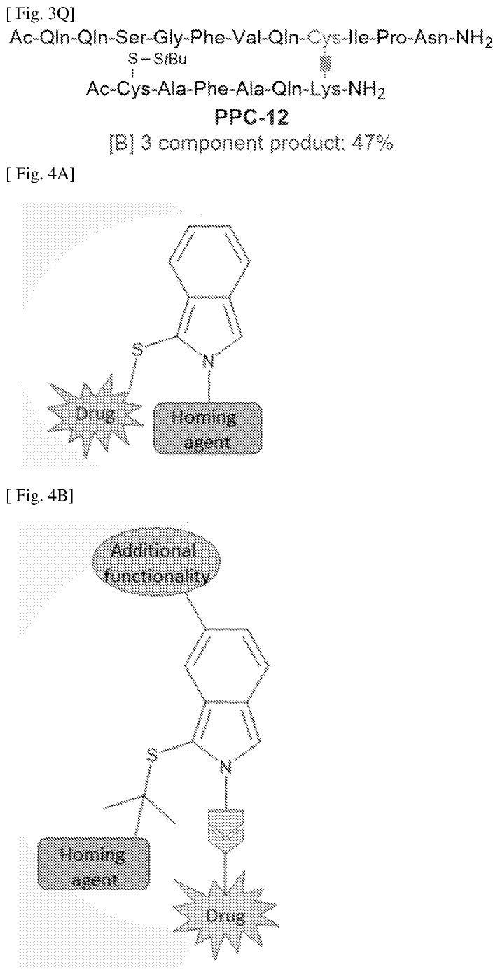

Twelve representative model peptide-peptide conjugates PPC 1-12 were constructed (FIGS. 3F-3Q). The side chain functionalities present in the unprotected peptides, including COOH (Glu, Asp), CONH₂ (Gln, Asn), OH (Ser, Thr, Tyr), guanidine (Arg), imidazole (His), and S-StBu (Cys) were fully compatible. According to the result, when no guanidine was added, the ratio of the two-component product to the isolated three-component product was 56:44 on average. When guanidine was added, only the three-component product could be isolated, and no two-component product was observed, or the amount was too minimal to be purified by the HPLC. Overall, this model study demonstrated the chemoselectivity and robustness of the chemistry, as well as its ability to be utilized as a facile approach to conjugate modular motifs, such as unprotected peptides.

Among the twelve representative entries, most of them only required 3 M of guanidine to show distinguished selectivity towards the desired three-component products. Yet, to achieve an enhanced reaction selectivity and efficiency, the concentration of guanidine added may be adjusted, due to the varied electrophilicity of imine and nucleophilicity of thiol in the intermediate.

With the successful peptide-peptide conjugate models in hand, it was hypothesized that this reaction could be further extended to prepare conjugates with active bio-properties, such as peptide-drug conjugates. To this end, maytansinoid (DM1) was selected as the cytotoxic agent. Although DM1 exhibits high antitumor activity, its clinical application is limited due to its significant systemic toxicity (Perrino, et al., Cancer Res. 2014, 74, 2569-2578). To address this issue, DM1 can be conjugated to a targeting agent. For example, Trastuzumab emtansine (T-DM1), a well-known antibody-conjugated DM1 derivative, was approved by the FDA in 2013 for the treatment of HER2-positive metastatic breast cancer (Amiri-Kordestani, et al., Clin. Cancer Res. 2014, 20, 4436-4441). In the present study, the free thiol group on DM1 makes it suitable for the OPA-amine-thiol three-component reaction.

Apart from the choice of drug, the selection of the therapeutic target is also a factor to be considered. Integrins, a family of cell surface receptors, are involved in a wide range of cell-extracellular matrix interactions, cell-cell interactions, and signal transduction (Hynes, Cell 1992, 69, 11-25; Cabodi, et al., Adv. Exp. Med. Biol. 2010, 674, 43-54). Among the twenty-four subtypes of integrins, αᵥβ₃ has attracted the most attention for three reasons. First, integrin αᵥβ₃ was found to be highly over-expressed in clinical glioblastoma and melanoma, thereby differentiating them from physiologically normal cells (Desgrosellier &Cheresh, Nat. Rev. Cancer 2010, 10, 9-22). Second, αᵥβ₃ plays an important role in cancer progression, tumor metastasis and angiogenesis (Desgrosellier &Cheresh, Nat. Rev. Cancer 2010, 10, 9-22). Third, integrin αᵥβ₃ is highly expressed on activated endothelial cells and new-born vessels, but it is absent in most normal organ systems and resting endothelial cells (Liu, et al. Drug Dev. Res. 2008, 69, 329-339). These characteristics make αᵥβ₃ an attractive integrin for tumor cell and tumor vasculature-targeted therapy.

The arginine-glycine-aspartic acid (RGD) cell adhesion sequence was discovered in fibronectin over two decades ago (Pierschbacher &Ruoslahti, Nature 1984, 309, 30-33; Ruoslahti &Pierschbacher, Science 1987, 238, 491-497). Later studies have revealed its strong binding affinity and selectivity to integrins, particularly to the αᵥ β₃ subtype (Chen &Chen, Theranostics 2011, 1, 189-200). On the atomic basis, this ligand binds at the major interface between the αv and β3 subunits and each residue participates extensively in this interaction (Xiong, et al., Science 2002, 296, 151-155). As this motif is highly conserved, it has been used as a vector to deliver drugs in active tumor-targeting therapy (Kapp, et al., Sci. Rep. 2017, 7, 39805). Comprehensive studies were conducted to optimize the structure of the RGD motif to further enhance its binding with the αᵥβ₃ integrin. Among the derivatives studied, the binding affinity of the cyclic peptides c [RGDfK] and c [RGDfV] have increased 39-fold and 58-fold, respectively, compared to the linear RGD tripeptide (Kapp, et al., Sci. Rep. 2017, 7, 39805). The free amine present on the Lys side chain of c [RGDfK] makes it a suitable reacting counterpart for the method of the present study. Overall, the OPA-c [RGDfK]-DM1 conjugate was used as an ideal test point for the current OPA-peptide-drug conjugation study.

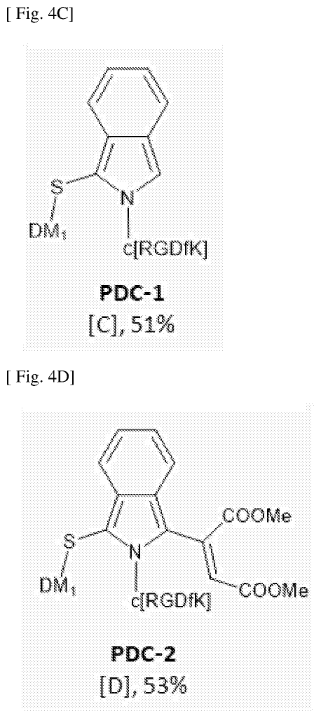

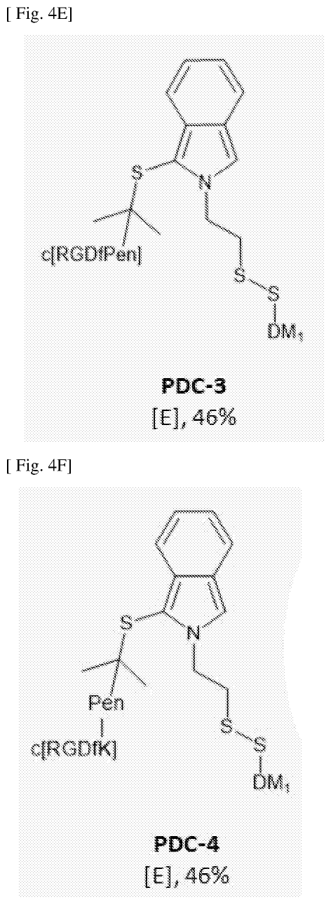

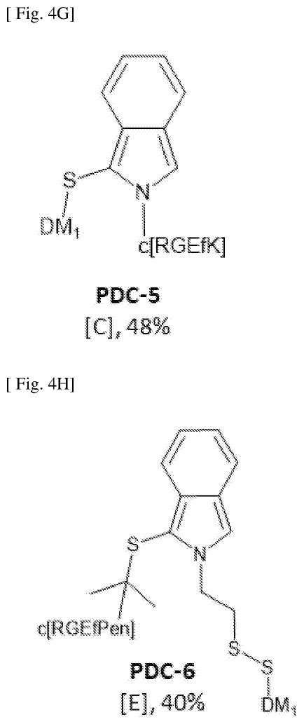

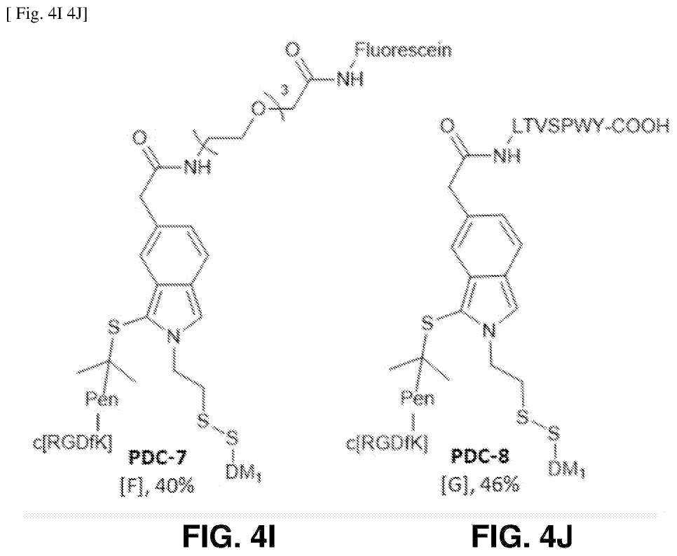

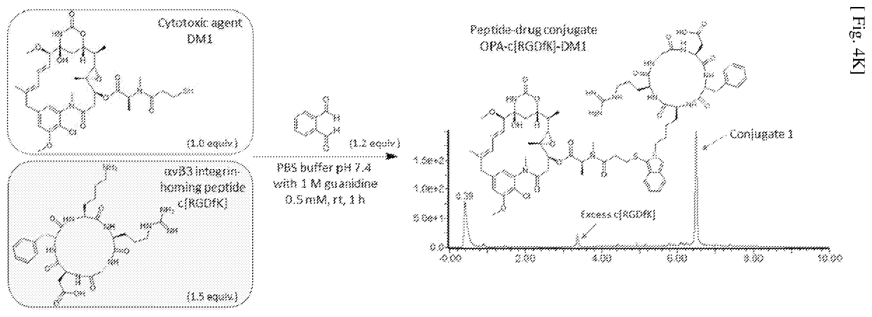

The improved conditions described above for the peptide-peptide conjugates was first applied in the synthesis of OPA-c [RGDfK]-DM1 with a reduced reaction condition at 0.5 mM to ensure complete dissolution of the drug. Under these calibrated conditions, the cytotoxic agent DM1 (1.0 equiv.) and integrin αᵥβ₃-targeting cyclic peptide c [RGDfK] (1.5 equiv.) reacted with OPA (1.2 equiv.) in PBS buffer with 1 M guanidine, at a 0.5 mM concentration. The reaction was completed in 1 hour, with excellent selectivity towards the three-component conjugate product and generated PDC-1 in 51% isolated yield (FIG. 4C).

Despite the smooth reaction, the resultant 1-thio-isoindole conjugate was not stable after HPLC purification. The resultant conjugate gradually decayed. The increased decomposition rate in acidic medium is consistent with the reported acid-catalyzed vinyl sulfide hydrolysis (Simons &Johnson, Annals in Biochemistry 1977, 82, 250-254). In addition, the resulting conjugate and the two-component by-product were prone to oxidation by air over time (Zuman, Chem. Rev. 2004, 104, 3217-3238).

To enhance the stability of the conjugate, two approaches were tested. In the first approach, the isoindole moiety was modified with dimethyl acetylenedicarboxylate (DMAC). The DMAC-isoindole adduct was reported to be more stable compared to isoindole (Zhang, et al., J. Am. Chem. Soc. 2019, 141, 12274-12279; Simons, et al., J. Org. Chem. 1981, 46, 4739-4744). Therefore, the OPA-c [RGDfK]-DM1-DMAC conjugate, PDC-2 (FIG. 4D) was prepared under condition [D] (Condition [C], one-point DMAC (2.0 equiv.), 1 h). In comparison to PDC-1, PDC-2 showed enhanced stability in aqueous solvents under air atmosphere over a wide pH range.

The second approach was to substitute the thiol group in the reaction with a more hindered thiol substrate, as it was hypothesized that a bulkier resultant conjugate might have enhanced stability. Considering that the Lys residue in c [RGDfK] was blocked for the conjugation, it was replaced by the penicillamine with little effects on its integrin-targeting ability. The cyclic peptide c [RGDfPen] was therefore prepared.

A triggered drug-releasing system was incorporated into the design, with the goal to maximize the intracellular level of free DM1 and improve its therapeutic outcome. This was accomplished via the use of reduction-sensitive linkers based on the highly reducing environment of the intracellular compartment, which contains 1000-fold higher glutathione (GSH) in the cytoplasm compared to the plasma (Meister & Anderson, Annu. Rev. Biochem. 1983, 52, 711-760). Moreover, the concentration of GSH in cancer cells was found to be even higher due to hypoxia (Bansal &Simon, J. Cell Biol. 2018, 217, 2291-2298). Redox-sensitive linkages, including the disulfide bond, have been targeted drug delivery systems (Austin, et al., Proc. Natl. Acad. Sci. U.S.A. 2005, 102, 17987-17992; Wang, et al., Curr. Med. Chem. 2012, 19, 2976-2983). Taking advantage of the free thiol group on DM1, a disulfide bond and an amine group were integrated into it for conjugation. A modified DM1, H₂NCH₂CH₂—S—S-DM1, was synthesized as a result.

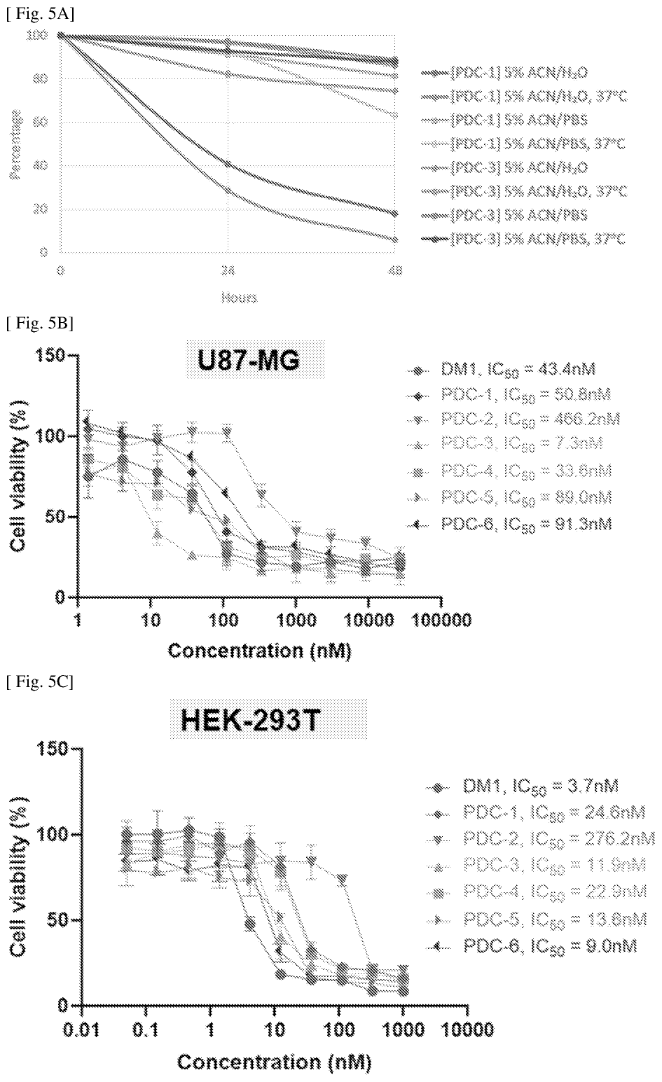

The OPA-amine-3°-thiol reaction with the two components was conducted. The reaction efficiency was much lower compared to the OPA-amine-thiol reaction due to steric hindrance. As the DM1 is now linked to a free amine and has improved solubility, it was hypothesized that the reaction may be carried out in higher concentrations. Eventually, it was observed that over 90% of the conversion could be achieved in 3 hours when a concentration of 5 mM was used. The condition [E] (3°-thiol (1.0 equiv.), OPA (1.2 equiv.) in PBS buffer pH 7.4 with 6 M guanidine, 5 mM, room temperature, 3 h) has afforded the desired product PDC-3 (FIG. 4E) in excellent selectivity and 46% isolated yield. The purified PDC-3 had improved stability compared to PDC-1 (FIG. 5A). Another conjugate utilizing Pen for the reaction was also designed. The goal was to minimize the structural modification on the c [RGDfK] targeting sequence, to fully preserve its activity. A cyclic peptide c [RGDf (K-Pen)] with Pen installed on the Lys side chain was synthesized and PDC-4 was generated with 46% isolated yield (FIG. 4F).

For biological assessment two conjugates were synthesized as negative controls. A single amino acid mutation Asp->Glu was incorporated in the RGD integrin-targeting motif, with minimal alteration on its structure and size. Although Asp and Glu confer a similar charge, the binding of the RGD tripeptide towards integrin is very specific and does not tolerate structural changes. As a result, the targeting property of the RGD motif was completely abolished when subjected to this modification (Brake, et al., J. Cell Biol. 1990, 111, 1275-1281; Mondal, et al., Biomaterials 2013, 34, 6249-6260). PDC-5 (FIG. 4G) and PDC-6 (FIG. 4H) containing the non-targeting control cyclic peptides c [RGEfK] and c [RGEfPen] were prepared according to conditions [C] (thiol (1.0 equiv.), amine (1.5 equiv.), OPA (1.2 equiv.) in PBS buffer pH 7.4 with 1 M guanidine, 0.5 mM, room temperature, 1 h) and [E] (3°-thiol (1.0 equiv.), OPA (1.2 equiv.) in PBS buffer pH 7.4 with 6 M guanidine, 5 mM, room temperature, 3 h). Overall, the synthesis of PDC 1-6 demonstrated the flexibility of the conjugation strategy of the current study with regard to the substrate scope (FIGS. 4C-4H). This reaction is not only compatible with the linear peptides, but is also compatible with cyclic peptides and drugs that bear an amine, thiol or a bulkier 3°-thiol group.

Next, the biological activities of these conjugates were evaluated. U87-MG was reported to express a high level of αᵥβ₃ integrin receptors and has been used as the positive control cell line for various RGD-containing targeted drugs, including Cilengitide (Maurer, et al., Neuro-Oncology 2009, 11, 747-756; Chen and Chen, Theranostics 2011, 1, 189-200). In contrast, the human embryonic kidney cell line HEK-293T that expresses a low level of αᵥβ₃ integrin served as the negative control (Hussain, et al., Nucleic Acid Ther. 2013, 23, 203-212).

Then, the in vitro antitumor efficacy of PDCs 1-6 was examined by CCK-8 assay (FIG. 5B). The IC₅₀ of free DM1 and its conjugates were compared internally within the same cell line, to determine the potency of the six compounds. In the tumor model U87-MG cell line, PDC-3 and PDC-4 showed a 5.95-fold and 1.29-fold increase in the cytotoxicity compared to free DM1 respectively. These results clearly demonstrate the contribution of the three factors to the improved activity: αᵥβ₃-targeting effect of RGD peptide, releasable cytotoxic reagent DM1, and improved conjugate stability. The functional conjugates can be facilely constructed by our three-component reaction conjugation method, and the stability improvement facilitated by 3°-thiol incorporation shows superiority than the non-tolerated isoindole core modification technique. For PDC-1, which bears the 1-thio-isoindole structure and a non-releasable DM1, cytotoxicity slightly decreased 0.85-fold. The decrease in activity is possibly due to the introduction of the RGD cyclic peptide on DM1, which may have hindered the drug from reaching the cells. For both negative controls, PDC-5 and PDC-6 that had the mutated RGE targeting motif, their antitumor efficacy decreased 0.49-fold and 0.48-fold respectively. The least active compound tested was PDC-2, which only showed 0.09-fold activity.

The peptide-drug conjugates were tested on the negative control cell line. In HEK-293T cells (FIG. 5C), the conjugates had reduced cytotoxicity, ranging from 0.01-fold to 0.41-fold of the activity of DM1. In general, no significant differences in the cytotoxicity of the RGD- or RGE-conjugates were observed. Although PDC-3 and PDC-6 carrying the releasable DM1 had relatively better activity among the six compounds, their cytotoxicity was still reduced compared to DM1. For PDC-3 and PDC-4 that had the best activity in U87-MG cells, their cytotoxicity in the αᵥβ₃-negative cell line reduced to only 0.31-fold and 0.16-fold respectively. This result illustrated that the targeting-peptide specifically delivered the drug to the U87-MG cells with higher expression of the αᵥβ₃ receptors. Additionally, PDC-2 also had the least cytotoxicity in HEK-293T cells. The combined results suggests that the DMAC-isoindole adduct scaffold is probably not the best core for biomedical applications.

In light of the encouraging cytotoxicity test results above, further experiments were performed employing other types of modular motifs and/or functionalities to the conjugate. Fluorescent-targeting peptide conjugates have been reported as powerful tools for non-invasive imaging in preclinical models (Cheng, et al., Bioconjug. Chemistry 2005, 16, 1433-1441). The tumor tissue can be visualized when the targeting peptide binds to its specific receptors. This imaging modality has made an impact on studying diseases at the molecular level. In addition, targeted imaging agents have also been adopted for the intracellular internalization studies (Garanger, et al., Mol. Ther. 2005, 12, 1168-1175).

These fluorescent and additional targeting compounds were used to further functionalize the OPA (FIGS. 41 and 4J). The OPA derivative OPA-CH2-COOH was first coupled with fluoresceinamine, generating an OPA-fluorophore linked by an amide bond. The reaction was conducted under the established conditions [C] (thiol (1.0 equiv.), amine (1.5 equiv.), OPA (1.2 equiv.) in PBS buffer pH 7.4 with 1 M guanidine, 0.5 mM, room temperature, 1 h) and [E] (3°-thiol (1.0 equiv.), OPA (1.2 equiv.) in PBS buffer pH 7.4 with 6 M guanidine, 5 mM, room temperature, 3 h). No product was observed. The significantly reduced reaction efficiency was possibly due to the steric hindrance of the OPA derivative and the 3°-thiol group. The hypothesized steric bulk issue was resolved by adding a PEG; linker between the OPA and fluorescein. Extensive screening revealed that the further-functionalized conjugate could be achieved under heating conditions, while the use of excess reactants could further accelerate the reaction. With the new condition [F] (3°-thiol (3.0 equiv.), amine (1.0 equiv.), functionalized OPA-fluorescein (3.0 equiv.) in PBS buffer pH 7.4 with 6 M guanidine, 1 mM 37° C., 2 h) in hand, PDC-7 (FIG. 4I) was prepared with 40% isolated yield. Next, U87-MG cells treated with PDC-7 were subjected to fluorescence confocal imaging, and internalization of the conjugate was observed. This further-functionalized conjugate incorporating OPA-PEG3-fluorescein can be used to study the internalization process of the drug conjugates generated. Additionally, derivatization of OPA with a near-infrared fluorochrome can be employed to generate conjugates applicable to in vivo imaging (Cheng, et al., Bioconjug. Chem. 2005, 16, 1433-1441).

Apart from the fluorophore, it was assessed whether OPA could be linked to other functionalities. The human epidermal growth factor receptor 2 (HER2) is overexpressed in various human cancers, such as breast cancer and ovarian cancer. HER2 plays an essential role in the proliferation and anti-apoptosis mechanisms, thereby making it an important diagnostic and therapeutic target (Tai, et al., J. Control. Release 2010, 146, 264-275). The peptide LTVSPWY identified via phage display was reported to bind HER2 receptors (Pero, et al., Int. J. Cancer Res. 2004, 111, 951-960) 42. The corresponding OPA-peptide derivative was synthesized. Unlike the challenging reactions with OPA-fluorescein, the OPA-peptide showed better reactivity, possibly due to its less congested structure. Utilizing reaction condition [G] (3°-thiol (2.0 equiv.), amine (1.0 equiv.), functionalized OPA-peptide (1.2 equiv.) in PBS buffer pH 7.4 with 6 M guanidine, 1 mM room temperature, 2 h), the dual-targeting peptides conjugate PDC-8 (FIG. 4J) was constructed with a 46% isolated yield.

These further-functionalized conjugates synthesized via the OPA derivatives further expanded the scope of the chemistry of the compounds. These examples demonstrate that the extra functionality is highly versatile, with both fluorophore and peptide being compatible with the established conjugation reaction. Although the additional functionality may impose steric bulk on the dialdehyde, this drawback was overcome by introducing a spacer as described above. Moreover, the reaction efficiency could be increased by adding more reactants or heating the reaction at 37° C.

### SUMMARY

In summary, the intermolecular OPA-amine-thiol reaction that has been used for fluorometric detection of α-amino acids was re-tested under new conditions. As compared with the conventional conditions which require a large excess amount of thiol (e.g., 10-3000 equiv.), it is demonstrated that an additive (such as guanidine or sodium bisulfite) served as an effective additive to realize the stoichiometric reaction.

This method provides an effective strategy for conjugation and to construct complex molecular structures in a modular fashion. Furthermore, via introducing additional functionality on the OPA, the structural and functional complexity of the resulting conjugate can be further diversified.

A set of conjugates (such as peptide-peptide conjugates and peptide-drug conjugates) have been constructed, including RGD-DM1 conjugates. Besides, it is worth noting that the 1-thio-isoindole products from OPA-amine-thiol reaction are less stable. This problem was solved by using sterically hindered thiol substrate, such as replacing cysteine with penicillamine, or generating a 1-thio-isoindole core that contained a further substituent on the pyrrole ring. From the bioactivity studies on U87-MG cells that express upregulated αᵥβ₃ integrins, two RGD-DM1 conjugates containing the 3°-thiol moieties and releasable drug design have resulted in up to 5.95-fold improvement in cytotoxicity compared to the free DM1.

The data show that these reactions are highly chemoselective. Further, the isolated yields for the peptide-peptide conjugates and peptide-drug conjugates ranged from 40-53%. The method is broadly applicable on various substrates, including, but not limited to, linear or cyclic peptides, drugs, and fluorophores. Finally, via the use of a pre-modified reactant (such as an OPA derivative), the structural and functional complexities of the resultant conjugate can be further diversified.

Those skilled in the art will recognize, or be able to ascertain using no more than routine experimentation, many equivalents to the specific embodiments of the method and compositions described herein. Such equivalents are intended to be encompassed by the following claims.

1. A method of making a conjugate, the method comprising:
reacting intermolecularly a first reactant, a second reactant, and a third reactant in the presence of an additive to form the conjugate, preferably wherein the additive suppresses a product formed by a reaction between only the first reactant and the third reactant,
wherein:
the first reactant comprises a structure: 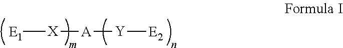
the second reactant comprises a nucleophile denoted nu₁,
the third reactant comprises a nucleophile denoted nu₂,
preferably, the second reactant and the third reactant are not covalently linked prior to the reaction,
nu₁ and nu₂ are preferably selected from thiol groups, amine groups, and thiolate ions, preferably wherein nu₁ and nu₂ are not the same,
m and n are independently integers from 1 to 5, such as 1, 2, 3, 4, or 5, preferably, m and n are 1,
A is a substituted aryl, an unsubstituted aryl, a substituted heteroaryl, an unsubstituted heteroaryl, a substituted polyaryl, an unsubstituted polyaryl, a substituted polyheteroaryl, an unsubstituted polyheteroaryl,
a substituted cycloalkyl, an unsubstituted cycloalkyl, a substituted cycloalkenyl, an unsubstituted cycloalkenyl, a substituted cycloalkynyl, an unsubstituted cycloalkynyl, a substituted C₁-C₂₀ heterocyclyl, an unsubstituted C₁-C₂₀ heterocyclyl, or a fused combination thereof, such as substituted aryl fused with substituted heteroaryl, substituted aryl fused with unsubstituted heteroaryl, unsubstituted aryl fused with substituted heteroaryl, or substituted aryl fused with unsubstituted heteroaryl,
X and Y are independently absent, substituted alkyl, unsubstituted alkyl, substituted alkenyl, unsubstituted alkenyl, substituted alkynyl, unsubstituted alkynyl, unsubstituted alkoxy, substituted alkoxy, unsubstituted aroxy, substituted aroxy, unsubstituted alkylthio, substituted alkylthio, unsubstituted carbonyl, substituted carbonyl, unsubstituted carboxyl, substituted carboxyl, unsubstituted ester, substituted ester, unsubstituted ether, substituted ether, unsubstituted polyether, substituted polyether, substituted amino, unsubstituted amino, substituted amide, unsubstituted amide, substituted aryl, unsubstituted aryl, substituted heteroaryl, unsubstituted heteroaryl, substituted polyaryl, unsubstituted polyaryl, substituted polyheteroaryl, unsubstituted polyheteroaryl, substituted C₃-C₂₀ cycloalkyl, unsubstituted C₃-C₂₀ cycloalkyl, substituted C₁-C₂₀ heterocyclyl, unsubstituted C₁-C₂₀ heterocyclyl, substituted C₃-C₂₀ cycloalkenyl, unsubstituted C₃-C₂₀ cycloalkenyl, substituted C₃-C₂₀ cycloalkynyl, unsubstituted C₃-C₂₀ Cycloalkynyl, or fused combinations thereof, and
E₁ and E₂ are electrophiles independently selected from substituted carbonyl or unsubstituted carbonyl, such as aldehydes, ketones, Michael acceptors; substituted carboxyl; or unsubstituted carboxyl.

2. The method of claim 1, wherein the additive comprises a nucleophile, preferably selected from guanidines, ureas, thioureas, sodium bisulfite, and combinations thereof.

3. The method of claim 1 or 2, wherein the concentration of the additive is between about 0.5 M and about 10 M, between about 1 M and about 8 M, or between about 3 M and about 8 M, such as 1 M, 2 M, 3 M, 4 M, 5 M, 6 M, 7 M, 8 M, 9 M, or 10 M, preferably 1 M, 3 M, 6 M, and 8 M.

4. The method of any one of claims 1 to 3, wherein the mole ratio of the first reactant to the second reactant is between 1:8 to 8:1, between 1:5 to 5:1, between 1:4 to 4:1, or between 1:3 to 3:1, such as 1:1.2, 1:1, or 2:1.2.

5. The method of any one of claims 1 to 4, wherein the mole ratio of the second reactant to the third reactant is between 1:4 to 4:1 or between 1:3 to 3:1, such as 1:1, 1:1.5, 3:1, or 2:1.

6. The method of any one of claims 1 to 5, wherein the second reactant and the third reactant are independently therapeutic agents, prophylactic agents, diagnostic agents, or targeting agents.

7. The method of any one of claims 1 to 6, wherein the second reactant and the third reactant are independently linear peptides, cyclic peptides, non-polymeric small molecule drugs (such as those having a molecular weight between 100 Da and 2, 500 Da), fluorophores, chemiluminescent compounds, or a combination thereof.

8. The method of any one of claims 1 to 7, wherein the second reactant comprises a thiol group that reacts with the first reactant.

9. The method of any one of claims 1 to 8, wherein the second reactant comprises a sterically hindered thiol group that reacts with the first reactant.

10. The method of any one of claims 1 to 9, wherein the third reactant comprises an amine group that reacts with the first reactant.

11. The method of any one of claims 1 to 10, wherein reacting the first reactant, second reactant, and third reactant forms the conjugate comprising a substituted 1-thio-isoindole moiety or an unsubstituted 1-thio-isoindole moiety.

12. The method of any one of claims 1 to 11, wherein the first reactant has a structure: 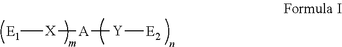
wherein:
A is a substituted aryl, an unsubstituted aryl, a substituted heteroaryl, an unsubstituted heteroaryl, a substituted polyaryl, an unsubstituted polyaryl, a substituted polyheteroaryl, an unsubstituted polyheteroaryl, a substituted C₁-C₂₀ heterocyclyl, an unsubstituted C₁-C₂₀ heterocyclyl, or a fused combination thereof.

13. The method of any one of claims 1 to 12, wherein for Formula I, A is a substituted aryl or an unsubstituted aryl.

14. The method of any one of claims 1 to 13, wherein the first reactant has a structure:

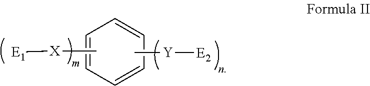

15. The method of any one of claims 1 to 14, wherein m and n are 1.

16. The method of any one of claims 1 to 15, wherein the first reactant has a structure:

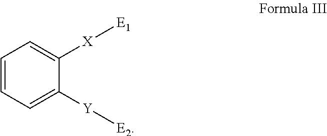

17. The method of any one of claims 1 to 16, wherein the first reactant has a structure:

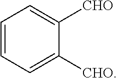

18. The method of any one of claims 1 to 17, wherein the reaction is performed in an aqueous buffer.

19. The method of any one of claims 1 to 18, comprising mixing the additive with a suitable buffer, such as phosphate-buffered saline (PBS), to form a solution prior to adding the first, second, and third reactants.

20. The method of any one of claims 1 to 19, comprising adding the first reactant and the second reactant in a solution containing the additive and a suitable buffer, prior to adding the third reactant.

21. The method of claim 19 or 20, wherein the buffer has a pH between about 7.0 and about 7.5.

22. The method of any one of claims 1 to 21, wherein the reaction is performed at room temperature, such as between about 23° C. and about 37° C., or about 23° C. and about 28° C.

23. The method of any one of claims 1 to 22, wherein the reaction has a concentration between about 0.1 mM and about 7.5 mM, between about 0.1 mM and about 5 mM, between about 0.1 mM and about 2.5 mM, between about 0.1 mM and about 2 mM, between about 0.5 mM and about 2.5 mM, or between about 0.5 mM and about 2 mM.

24. The method of any one of claims 1 to 23, comprising:
(i) adding the additive in a suitable buffer, such as PBS, to form a solution, dispersion, or suspension,
(ii) adding the first reactant and the second reactant in the solution, dispersion, or suspension of step (i) to form another solution, dispersion, or suspension, and
(iii) adding the third reactant to the solution of step (ii) to form another solution, dispersion, or suspension.

25. The method of claim 24, wherein the solution, dispersion, or suspension of step (iii) is maintained in an air atmosphere and at a temperature between about 23° C. and about 40° C., between about 23° C. and about 28° C., or between about 30° C. and about 40° C., to form the conjugate.

26. A conjugate having a structure: 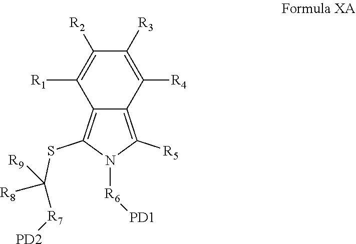
wherein:
R₁-R₄ are independently hydrogen, unsubstituted C₁-C₁₀ alkyl, substituted C₁-C₁₀ alkyl, unsubstituted alkoxy, substituted alkoxy, unsubstituted aroxy, substituted aroxy, unsubstituted alkylthio, substituted alkylthio, unsubstituted carbonyl, substituted carbonyl, unsubstituted carboxyl, substituted carboxyl, unsubstituted ester, substituted ester, substituted amino, unsubstituted amino, substituted amide, unsubstituted amide, substituted aryl, unsubstituted aryl, substituted heteroaryl, unsubstituted heteroaryl, substituted polyaryl, unsubstituted polyaryl, substituted polyheteroaryl, a unsubstituted polyheteroaryl, substituted cycloalkyl, unsubstituted cycloalkyl, substituted cycloalkenyl, unsubstituted cycloalkenyl, substituted cycloalkynyl, unsubstituted cycloalkynyl, substituted C₁-C₂₀ heterocyclyl, unsubstituted C₁-C₂₀ heterocyclyl, or fused combination thereof,
R₅ is hydrogen, unsubstituted C₁-C₁₀ alkyl, substituted C₁-C₁₀ alkyl, unsubstituted C₁-C₁₀ alkenyl, substituted C₁-C₁₀ alkenyl, unsubstituted alkoxy, substituted alkoxy, unsubstituted aroxy, substituted aroxy, unsubstituted alkylthio, substituted alkylthio, unsubstituted carbonyl, substituted carbonyl, unsubstituted carboxyl, substituted carboxyl, unsubstituted ester, substituted ester, substituted amino, unsubstituted amino, substituted amide, unsubstituted amide, substituted aryl, unsubstituted aryl, substituted heteroaryl, unsubstituted heteroaryl, substituted polyaryl, unsubstituted polyaryl, substituted polyheteroaryl, a unsubstituted polyheteroaryl, substituted cycloalkyl, unsubstituted cycloalkyl, substituted cycloalkenyl, unsubstituted cycloalkenyl, substituted cycloalkynyl, unsubstituted cycloalkynyl, substituted C₁-C ₂₀ heterocyclyl, unsubstituted C₁-C₂₀ heterocyclyl, or fused combination thereof,
R₆ and R⁷ are independently unsubstituted C₁-C₁₀ alkyl, substituted C₁-C₁₀ alkyl, substituted aryl, unsubstituted aryl, substituted heteroaryl, unsubstituted heteroaryl, substituted polyaryl, unsubstituted polyaryl, substituted polyheteroaryl, a unsubstituted polyheteroaryl, substituted cycloalkyl, unsubstituted cycloalkyl, substituted cycloalkenyl, unsubstituted cycloalkenyl, substituted cycloalkynyl, unsubstituted cycloalkynyl, substituted C₁-C₂₀ heterocyclyl, unsubstituted C₁-C₂₀ heterocyclyl, or fused combination thereof, with the proviso that R₆ and R₇ are at least divalent, as valency permits,
R₈ and R₉ are independently hydrogen, unsubstituted C₁-C₁₀ alkyl, substituted C₁-C₁₀ alkyl, with the proviso that at least one of R₈ and R₉ is not hydrogen, such as unsubstituted C₁-C₁₀ alkyl, or substituted C₁-C₁₀ alkyl, and
PD1 and PD2 independently comprise a peptide, non-peptide therapeutic agent, non-peptide diagnostic agent, non-peptide prophylactic agent, or non-peptide targeting agent.

27. The conjugate of claim 26, wherein R¹-R₄ are independently hydrogen, unsubstituted C₁-C₁₀ alkyl, or substituted C₁-C₁₀ alkyl.

28. The conjugate of claim 26 or 27, wherein R¹-R₄ are hydrogen.

29. The conjugate of claim 26 or 27, wherein at least one of R₁-R₄ independently comprises a targeting agent, diagnostic agent, prophylactic agent, or a therapeutic agent, preferably wherein the targeting agent contains a peptide.

30. The conjugate of any one of claims 26 to 29, wherein R₅ is hydrogen, unsubstituted C₁-C₁₀ alkyl, substituted C₁-C₁₀ alkyl, unsubstituted C₁-C₁₀ alkenyl, or substituted C₁-C₁₀ alkenyl.

31. The conjugate of any one of claims 26 to 30, wherein R⁶ and R⁷ are independently unsubstituted C₁-C₁₀ alkyl, or substituted C₁-C₁₀ alkyl, with the proviso that R₆ and R⁷ are at least divalent, as valency permits.

32. The conjugate of any one of claims 26 to 31, wherein R⁶ and R⁷ independently comprise a stimuli-responsive chemical moiety, preferably wherein R⁶ contains a stimuli-responsive chemical moiety.

33. The conjugate of any one of claims 26 to 32, wherein R₈ and R₉ are independently unsubstituted C₁-C₁₀ alkyl or substituted C₁-C₁₀ alkyl.

34. The conjugate of any one of claims 26 to 33, wherein PD1 and PD2 comprise a peptide, preferably an unprotected peptide.

35. The conjugate of any one of claims 26 to 34, wherein one of PD1 and PD2 comprises a peptide, preferably an unprotected peptide, and the other of PD1 and PD2 comprises a non-peptide therapeutic agent, non-peptide diagnostic agent, non-peptide prophylactic agent, or non-peptide targeting agent.

36. The conjugate of any one of claims 26 to 35, wherein one of PD1 and PD2 comprises a peptide, preferably an unprotected peptide, and the other of PD1 and PD2 comprises a non-peptide therapeutic agent.
